(19) United States
(12) Patent Application Publication
(10) Pub. No.: US 2023/0157587 A1
(43) Pub. Date: May 25, 2023

(54) A WEARABLE PATCH FOR CONTINUOUS ANALYSIS OF SWEAT AT A NATURALLY SECRETING RATE

(71) Applicant: The Regents of the University of California, Oakland, CA (US)

(72) Inventors: Ali Javey, Lafayette, CA (US); Hnin Yin Yin Nyein, Berkeley, CA (US); Brandon Nguyen K. Tran, Garden Grove, CA (US)

(73) Assignee: The Regents of the University of California, Oakland, CA (US)

(21) Appl. No.: 17/919,983

(22) PCT Filed: Apr. 20, 2021
(86) PCT No.: PCT/US2021/028244

§ 371 Date: Oct. 19, 2022

Related U.S. Application Data
(60) Provisional application No. 63/013,315, filed on Apr. 21, 2020.

(51) Int. Cl. A61B 5/145; A61B 5/1477; A61B 5/00

(52) U.S. Cl. CPC A61B 5/14517; A61B 5/14546; A61B 5/1477; A61B 5/6833

(57) ABSTRACT

In certain embodiments a microfluidic patch is provided that allows continuous analysis of natural sweat at various body locations of sedentary individuals. In certain embodiments the patch provides integrated electrical sweat rate sensor and electrochemical sensors to enable simultaneous detection of sweat rate and compositions such as pH, Cl⁻, and levodopa. The patch can facilitate dynamic sweat analysis related to light physical activities, hypoglycemia-induced sweating, and levodopa sensing for Parkinson's disease management. The device enables routine analysis of natural sweat dynamics arising from different physical and physiological functions which cannot be realized by current wearable sweat sensors.

### CROSS-REFERENCE TO RELATED APPLICATIONS

This application claims priority to and benefit of U.S. Ser. No. 63/013,315, filed Apr. 21, 2020, which is incorporated herein by reference in its entirety for all purposes.

### STATEMENT OF GOVERNMENTAL SUPPORT

This invention was made with government support under Grant Number 1160494 awarded by the National Science Foundation. The government has certain rights in the invention.

### BACKGROUND

Wearable electronics have been developed that can be worn by a user to continuously and closely monitor an individual's activities, such as walking or running. Such wearable electronics may include physiological sensors configured to sense certain physiological parameters of the wearer, such as heart rate, as well as motion sensors, GPS, radios, and altimeters.

Many of these electronic devices can be worn on or mated with human skin to continuously and closely monitor an individual's activities without unduly interrupting or limiting those activities. Biosensors on these wearable electronics may play a significant role in realizing personable medicine due to the capability for real-time monitoring of an individual's physiological biomarkers. Nonetheless, commercially available conventional wearable sensors are only currently capable of tracking an individual's physical activities and vital signs (e.g., step count, heart rate, etc.). They fail to provide insight into the user's health state at molecular levels.

To gain such insight into health state at a molecular level, human sweat is an excellent candidate for detection and measurement because it contains physiologically and metabolically rich information that can be retrieved non-invasively. Sweat analysis is currently used for applications such as disease diagnosis, drug abuse detection, and athletic performance optimization. Unfortunately, the sample collection and analysis are conventionally performed separately, thereby failing to provide a real-time profile of sweat content secretion, while requiring extensive lab analysis using bulky, and often expensive, instruments.

Development of wearable sweat biosensors has recently been explored where a variety of biosensors were used to measure analytes of interest. For example, U.S. Patent Application Publication No. US 2018/0263539 discloses a wearable sensing platform that includes sensors and circuits to sense aspects of a user's state by analyzing bodily fluids, such as sweat and/or urine, and a user's temperature. As described therein, a sensor array senses a plurality of different body fluid analytes, optionally at the same time. A signal conditioner is coupled to the sensor array. An interface is configured to transmit information corresponding to the conditioned sensor signals to a remote computing device. The wearable sensing platform may include a flexible printed circuit board to enable the wearable sensing platform, or a portion thereof, to conform to a portion of the user's body.

Recent emergence of wearable sweat sensors provides a promising future for non-invasive assessment of health physiology. To date, sweat sensors utilize conventional sweat induction approaches such as exercise, chemical, and thermal stimulation to obtain quantifiable sweat samples for on-body analysis (see, e.g., Yang et al. (2019) Nat. Biotechnol. DOI:10.1038/s41587-019-0321-x; Parlak et al. (2018) Sci. Adv. 4(7), eaar2904; Lee et al. (2017) Sci Adv. 3(3): e1601314; Yokus et al. (2020) Biosens. Bioelectron. 153: 112038; Emaminejad et al. (2017) Proc. Natl. Acad. Sci. USA, 114: 4625-4630; Jia et al. (2013) Anal. Chem. 85(14): 6553-6560; Kim et al. (2016) ACS Sens. 1(8): 1011-1019; Nyein et al. (2019) Sci. Adv. 5(8): eaaw9906; Alizadeh et al. (2018) Lab Chip, 18: 2632-2641; Bandodkar et al. (2019) Annu. Rev. Anal. Chem. 12: 1-22; Li et al. (2019) Small, 1903822; Koh et al. (2106) Sci. Trans. Med. 8(366): 366ra165; Twine et al. (2108) Lab Chip 18: 2816-2825; and the like). While these methods can provide large quantity of sweat in a short time (˜>2 μL cm⁻² in 15 mins), (Hussain et al. (2017) Clin. Biochem. Rev. 38(1): 13-34) they require artificial sweat induction to enable sweat analysis. These types of sweat may not be suitable in all applications. Naturally secreting sweat is an under-utilized source that excretes voluntarily even when individuals are at rest (Hu et al. (2018) Br. J. Dermatol. 178(6): 1246-1256) and offers many promising applications and clinical interests. Natural sweat rate in infants is closely related to defects of the central nervous system and emotional sweating (Foster et al. (1971) Arch. Dis. Child. 46: 444-451; Harpin & Rutter (1982) Arch. Dis. Child 57: 691-695). It is associated with the cerebral cortex activity and is correlated with severity of paresis in patients with brain infarction (Satoh et al. (1965) Jpn. J. Physiol. 15: 523-531; Korpelainen (1993) Neurology, 43: 1211-1214). It is also linked to physiological habituation of soldiers to combat experiences (Wood et al. (2009) Mil. Med. 174: 1215-1222). Patients with underlying medical conditions such as autonomic dysfunctions such as diabetes, cerebrovascular diseases, and Parkinson's disease are also accompanied by abnormalities in sweat rate (Cheshire et al. (2003) Sem. Neurol. 23(4): 399-406). Additionally, natural sweat secretes at a slow rate, allowing enough time for biochemicals to permeate and partition between blood and sweat, and to achieve equilibrium conditions between these fluid compartments (Sonner et al. (2015) Biomicrofluidics, 9(3): 031301). Therefore, natural sweat compositions may provide a close relation with blood biomarkers.

Despite its promising applications and clinical interests, an inherent inaccessibility of natural sweat has hindered our capability to utilize its rich information for diverse physiological monitoring. Natural sweat generally secretes at a significantly lower rate (˜10 nL min⁻¹ cm⁻²) than actively induced sweat (>250 nL min⁻¹ cm⁻²) and evaporates quickly (Hussain et al. (2017) Clin. Biochem. Rev. 38(1): 13-34; Taylor et al. (2913) Extrem. Physiol. Med. 2: 4). To address this limitation, natural sweat analyses were previously conducted through sampling sweat on interfaces like wet absorbent pad and hydrogel. These methods utilized diffusion of sweat chemicals from the skin into the interface and allowed analytes accumulation over a period of time for detectable signals (Kintz et al. (2000) J. Anal. Toxicol. 24: 557-561; Leggett et al. (2007) Angew. Chem. Int. Ed. 46: 4100-4103; Lin et al. (2019) ACS Sens. DOI: 10.1021/acssensors.9b01727). However, they do not allow monitoring temporal changes in sweat compositions. Low, stimulated sweat composition analyses were previously demonstrated using nafion and thiol derivatives as wicking media (Twine et al. (2108) Lab Chip 18: 2816-2825; Hauke et al. (2018) Lab Chip 18: 3750-3759; Lee et al. (2016) Nat. Nanotechol. 11: 566-572). They could neither collect sweat nor provide sweat rate. Continuous natural sweat rate analyses have traditionally been done in the hospital by monitoring humidity changes on the skin in a capsule such as in autonomic testing (Illigens & Gibbons (2009) Clin. Auton. Res. 19 (2), 79-87). Nevertheless, the use of bulky instrumentations for these sweat analyses has restricted the applications to clinical settings. The challenge remains in devising a wearable device that allows effective natural sweat capture and analyzes continuous sweat profile for routine assessment.

### SUMMARY

In various embodiments a microfluidic patch is provided that allows continuous analysis of natural sweat at various body locations of sedentary or active individuals. By modelling sweat glands and microfluidics according to the Poiseuille's law, in certain embodiments devices are provided comprising microchannels interfaced with a hydrophilic filler that can detect sweat rate down to 2 nL min⁻¹ cm⁻² even at the lowest secretion regions like wrist within an hour of device application. In certain embodiments the device is integrated with an electrical sweat rate sensor and electrochemical sensors to enable simultaneous detection of sweat rate and compositions such as pH, Cl—, levodopa, and the like. In certain embodiments the devices provide for dynamic sweat analysis related to light physical activities, hypoglycemia-induced sweating, and levodopa sensing for Parkinson's disease management. The device enables routine analysis of natural sweat dynamics arising from different physical and physiological functions that cannot be realized by current wearable sweat sensors. This can facilitate new sweat investigations related to individuals' well-being such as infant care, stroke rehabilitation, psychiatric assessment, and soldier welfare.

Accordingly, various embodiments provided herein may include, but need not be limited to, one or more of the following:

Embodiment 1: A wearable biometric monitoring system comprising:”
a hydrophilic material 106;
a sensing electrode 104; and
a microfluidic channel 110 connecting said hydrophilic material and said sensing electrode.

Embodiment 2: The wearable biometric monitoring system of embodiment 1, wherein device comprises a collection well 108 in fluid communication with said microfluidic channel and said hydrophilic material 106 is disposed in said collection well.

Embodiment 3: The wearable biometric monitoring system according to any one of embodiments 1-2, wherein said collection well provides a collection area ranging in diameter from about 1 mm to about 20 mm, or from about 2 mm up to about 10 mm, or from about 3 mm up to about 7 mm.

Embodiment 4: The wearable biometric monitoring system according of embodiment 3, wherein said collection well provides a collection area of about 8 mm.

Embodiment 5: The wearable biometric monitoring system according of embodiment 3, wherein said collection well provides a collection area of about 5 mm.

Embodiment 6: The wearable biometric monitoring system according of embodiment 3, wherein said collection well provides a collection area of about 3 mm.

Embodiment 7: The wearable biometric monitoring system according to any one of embodiments 1-6, wherein said hydrophilic material is laminated and includes hydrogel 204.

Embodiment 8: The wearable biometric monitoring system according to any one of embodiments 1-7, wherein said hydrogel comprises an agarose-glycerol (AG-GLY) hydrogel.

Embodiment 9: The wearable biometric monitoring system according to any one of embodiments 1-8, wherein said hydrophilic material comprises a hydrophilic polymer disposed on a patterned substrate.

Embodiment 10: The wearable biometric monitoring system of embodiment 9, wherein said hydrophilic polymer comprises polyvinyl alcohol (PVA).

Embodiment 11: The wearable biometric monitoring system according to any one of embodiments 1-10, wherein said patterned substrate comprises a patterned epoxy substrate.

Embodiment 12: The wearable biometric monitoring system of embodiment 11, wherein said substrate comprises a patterned SU8 substrate.

Embodiment 13: The wearable biometric monitoring system according to any one of embodiments 1-12, wherein said hydrophilic material comprises laminated substrate comprising a hydrophilic polymer disposed on a patterned substrate that is coated with a hydrophilic polymer.

Embodiment 14: The wearable biometric monitoring system according to any one of embodiments 1-13, wherein said microfluidic channel has a length of less than 33 cm, or less than 30 cm, or less than 25 cm, or less than 20 cm, or about 15 cm or less.

Embodiment 15: The wearable biometric monitoring system according to any one of embodiments 1-14, wherein said microfluidic channel has a minimum volume of about 750 nL.

Embodiment 16: The wearable biometric monitoring system according to any one of embodiments 14-15, wherein said microfluidic channel has a length of about 15 cm or less.

Embodiment 17: The wearable biometric monitoring system according to any one of embodiments 1-16, wherein said microfluidic channel has dimensions that provide a flow rate drop of less than about 10% along the length of said microfluidic channel.

Embodiment 18: The wearable biometric monitoring system according to any one of embodiments 1-17, wherein said microfluid channel has a cross-section area at least about 2,209 μm² (e.g., 47 μm×47 μm), or at least about 3600 μm², or at least about 4900 μm² (e.g., 70 μm×70 μm), or at least about 700 μm², or at least about 14,000 μm² (e.g., 200 μm×70 μm).

Embodiment 19: The wearable biometric monitoring system of embodiment 18, wherein said microfluidic channel has a cross-section area of about 70 μm×70 μm.

Embodiment 20: The wearable biometric monitoring system of embodiment 18, wherein said microfluid channel has a cross-section area of about 200 μm×70 μm.

Embodiment 21: The wearable biometric monitoring system according to any one of embodiments 1-20, wherein said sensing electrode(s) 104 are configured to be in fluid communication with a fluid in said microfluidic channel.

Embodiment 22: The wearable biometric monitoring system of embodiment 21, wherein said sensing electrodes 104 are configured to be aligned with the microfluidic channel 110.

Embodiment 23: The wearable biometric monitoring system according to any one of embodiments 21-22, wherein said sensing electrodes 104 are configured as two interdigitated wheel-shaped electrodes aligned with the microfluidic channel 110.

Embodiment 24: The wearable biometric monitoring system according to any one of embodiments 21-23, where said sensing electrodes comprise sweat rate sensing electrode(s) 104a and analyte detecting electrodes 104b.

Embodiment 25: The wearable biometric monitoring system of embodiment 24, wherein said sweat rate sensing electrodes 104a comprise radial conductive electrodes 104a1.

Embodiment 26: The wearable biometric monitoring system according to any one of embodiments 24-25, wherein said analyte detecting electrodes 104b comprise one or more regions 104b1 functionalized for detection of pH and/or an analyte.

Embodiment 27: The wearable biometric monitoring system of embodiment 26, wherein said analyte detecting electrode(s) 104b are functionalized for detection and/or quantification of an analyte selected from the group consisting of a metabolite, a drug, ethanol, a metal ion, and a salt.

Embodiment 28: The wearable biometric monitoring system according to any one of embodiments 1-27, wherein sensing electrode 104 is configured to measure sweat rate.

Embodiment 29: The wearable biometric monitoring system according to any one of embodiments 1-28, wherein sensing electrode 104 is configured to measure pH, Cl⁻, and/or levodopa.

Embodiment 30: The wearable biometric monitoring system of embodiment 29, wherein said system measures pH.

Embodiment 31: The wearable biometric monitoring system of embodiment 29, wherein said system measures Cl⁻.

Embodiment 32: The wearable biometric monitoring system of embodiment 29, wherein said system measures levodopa.

Embodiment 33: The wearable biometric monitoring system according to any one of embodiments 1-32, wherein said system is configured for detection by detection and/or quantification of electrical current or electrical potential.

Embodiment 34: The wearable biometric monitoring system according to any one of embodiments 1-33, wherein said microfluidic channel 110 is disposed in a microfluidic chip 102.

Embodiment 35: The wearable biometric monitoring system according to any one of embodiments 1-20, wherein said device is disposed on a flexible substrate 112.

Embodiment 36: The wearable biometric monitoring system of embodiment 35, wherein said substrate a flexible polymer.

Embodiment 37: The wearable biometric monitoring system of embodiment 36, wherein said substrate comprises polyethylene terephthalate (PET).

Embodiment 38: The wearable biometric monitoring system according to any one of embodiments 1-37, wherein said wearable biometric monitoring system comprises a skin adhesive 114 compatible with application to the skin.

Embodiment 39: The wearable biometric monitoring system of embodiment 38, wherein said skin adhesive 114 is disposed so that when said device is attached to the skin of a subject, said collection well is juxtaposed against a surface of said skin.

Embodiment 40: A wearable patch for analysis of a user's sweat comprising:
skin adhesive;
a microfluidic chip with a hydrophilic material and a microfluidic channel;
a sensing electrode;

wherein said skin adhesive is capable of attaching said microfluidic chip to the skin of a user and said hydrophilic material is capable of drawing sweat from said user so that said sweat can be transported into said microfluidic channel and to said electrode for analysis.

Embodiment 41: The wearable patch of embodiment 6 wherein said sensing electrode measures sweat rate.

Embodiment 42: The wearable patch of embodiment 6 wherein said sensing electrode is an electrochemical sensor which senses pH, Cl⁻ and/or levodopa.

Embodiment 43: A method of analyzing a user's sweat comprising:
selecting a patch comprising a skin adhesive, a microfluidic chip with a hydrophilic material, a microfluidic channel and a sensing electrode;
using said adhesive to apply said patch to a user's skin; and
collecting sweat from said user by drawing sweat from said user's skin with said hydrophilic material and transporting said sweat to said sensing electrode through said microfluidic channel; and, using said sensing electrode to analyze said user's sweat.

Embodiment 44: A method of analyzing a subject's sweat, said method comprising:
providing a subject with a wearable biometric monitoring system according to any one of embodiments 1-39 attached to the surface of the skin of said subject; and
operating said monitoring system to analyze the sweat of said subject.

Embodiment 45: The method of embodiment 44, wherein said monitoring system is operated to detect the sweat rate of said subject.

Embodiment 46: The method according to any one of embodiments 44-45, wherein said monitoring system is operated to determine the pH of the sweat of said subject.

Embodiment 47: The method according to any one of embodiments 44-46, wherein said monitoring system is operated to detect an analyte in the sweat of said subject where said analyte is selected from the group consisting of a metabolite, a drug, ethanol, a metal ion, and a salt.

Embodiment 48: The method of embodiment 47, wherein said monitoring system is operated to detect and/or quantify pH, Cl⁻, and/or levodopa in the sweat of said subject.

Embodiment 49: The method of embodiment 48, wherein said monitoring system is operated to measure Cl⁻ in the sweat of said subject.

Embodiment 50: The method of embodiment 48, wherein said monitoring system is operated to measure levodopa in the sweat of said subject.

Embodiment 51: The method according to any one of embodiments 44-50, wherein said subject is a human.

Embodiment 52: The method according to any one of embodiments 44-50, wherein said subject is a non-human mammal.

### BRIEF DESCRIPTION OF THE DRAWINGS

FIG. 1, schematically illustrates components of one embodiment of a microfluidic sweat analysis patch.

FIG. 2, schematically illustrates components of one embodiment of a hydrophilic filler (spacer). The illustrated filler a patterned epoxy mole mold covered with a hydrophilic polymer film (e.g., polyvinyl alcohol (PVA) film) and a hydrogel (e.g., agarose-glycerol (AG-GLY) hydrogel) and is embedded inside the collection well.

FIG. 3, schematically illustrates an electrode configuration for one embodiment of a microfluidic sweat analysis patch.

FIG. 4, panels a-e, shows a schematic of the design, structure, and usage of the microfluidic sweat analysis patch. Panel a) The patch contains multiple layers. It interfaces the skin via a skin adhesive and sweat is collected by assistance of hydrophilic filler into the microfluidics and eventually measured using sensing electrodes fabricated on a thin PET. Panel b) The hydrophilic filler includes a patterned SU8 mold covered with PVA film and AG-GLY hydrogel and is embedded inside the collection well. The filler enhances sweat collection by lowering sweat secretion pressure and taking up volume of the well otherwise will need to be filled. Panel c) An optical image of the sweat patch on a user's finger is displayed. Panel d) The patch can be worn on various locations and is used to monitor sweat dynamics without interrupting routine activities. Panel e) It can continuously monitor both sweat secretion rate and compositions for long-term without external sweat stimulation, as schematically shown using model trends.

FIG. 5, panels a-d, illustrates collection of at-rest thermoregulatory sweat on various parts of the body. Panel a) Sweat patches were placed on 8 different locations including shoulder, chest, bicep, wrist, abdomen, finger, thigh, and calf. Panel b) The sweat patch used for collection and imaging is displayed. Panel c) The bar graph shows a subject's local average sweat rates of regions indicated in (panel a) based on optical tracking of sweat in the microchannel from images like those in (panel d). Panel d) Optical images of microfluidic sweat collection at different locations and times are displayed. Note that collection areas and microfluidic dimensions are different for each location. Two different dimensions discussed in the results section of Example 1 were utilized, and a collection well with diameter varied from 3 to 10 mm were used. Measured rates were normalized by the collection area.

FIG. 6, panels a-h, illustrates sweat sensor characterization. Panel a) An impedimetric sweat rate sensing electrodes for detection of secretion rate is illustrated. An admittance (reciprocal of impedance) pulse is measured upon fluid contacting each of the radial electrode. Panel b) Electrochemical sensors for compositional analysis are functionalized near the tip of the four semicircles. These sensors are embedded inside the microchannel. Panel c) Admittance responses to solution containing NaCl concentrations of 10, 50, 100, and 200 mM. Panel d) Incremental volume filled inside the microchannel with respect to time is plotted when 10 and 200 mM NaCl solutions are flowed at a constant rate of 250 nL/min. The incremental volume corresponds to additional fluid filled between two adjacent radial electrodes. Panel e) Input flow rate from syringe pump and measured flow rate from sweat rate sensor are compared. Performance of (panel f) pH, (panel g) Cl—, and (panel h) levodopa sensors are presented

FIG. 7, panels a-d, illustrates in-situ sweat analysis of a healthy volunteer while performing daily tasks. Panel a) The study was conducted to explore dynamic heart rate and sweat behaviors of a sedentary subject during routine activities such as talking, walking, eating, etc. Panel b) A subject wore the microfluidic patch and a heart rate monitor on the wrist, and heart rate and sweat rate were continuously monitored for 6 h. A subject wore the microfluidic patch on (panel c) finger and (panel d) wrist, and heart rate, sweat rate, sweat pH and Cl— were simultaneously measured. Sweat measurement began 10 min and 4 h after sweat secretion began on finger and wrist, respectively.

FIG. 8, panels a-b, illustrates a twenty-hour in situ sweat analysis to identify stress events among routine activities. Sweat is monitored on the fingertip of a healthy volunteer along with heart rate and ambient temperature as the mostly sedentary subject performed intervals of public speaking (panel a) during a live-streamed academic conference in Trial 1, and (panel b) while teaching a class in Trial 2. The stress-inducing intervals of public speaking are associated with elevated heart rate and a sharp increase in sweat rate.

FIG. 9, panels a-b, illustrates in situ sweat analysis for hypoglycemia-induced sweat analysis. Sweat secretion rate was measured along with heart rate and ISF glucose levels of a diabetic subject. Subject had insulin injection to lower glucose levels in (panel a) trial 1 and (panel b) trial 2.

FIG. 10, panels a-b, illustrates in situ sweat analysis to assist Parkinson's disease management on daily basis. A healthy subject wore the microfluidic patch on the finger and had broad beans intake of (panel a) 1 dose=100 g and (panel b) 2 doses=200 g during the measurement duration. (levodopa=L-dopa).

FIG. 11 illustrates geometric parameters and dimensions for one illustrative embodiment of the microfluidic patch.

FIG. 12 illustrates the structure and function of the hydrogel-filler stack in the sweat collection well.

FIG. 13, panels a-b, shows optical images of the microfluidic patch and filler component. Panel a) Image of the assembled patch with PDMS microfluidic and electrode layers. Panel b) Image of the SU8 filler after fabrication of PET backing substrate. The filler has grooves for sweat to flow through but is connected and held together due to overexposure of SU8 during lithography. The filler is peeled off the PET backing for insertion into the collection well.

FIG. 14, panels a-b, illustrates a sweat rate sensor for detection of resting sweat rate in a short period of time with assistance of the hydrophilic filler and detection of resting sweat rate as low as 2 nL min⁻¹. As shown, the sweat rate sensor can be used to detect (panel a) flow rate in a short period of time with assistance of the hydrophilic filler. The collection well of the illustrated sensor has a 3 mm diameter and can hold ˜2.8 μL of fluid. The flow rate corresponds to 1700 nL min⁻¹ cm⁻². The sensor can detect (panel b) flow rate as low as 2 nL min⁻¹. Flow rate is measured electrically via admittance between the spoked electrodes underlying the channel. Note that injection pumps set to nL min⁻¹ flow rates can generate pulsatile flow, causing some variations and transient effects in flow rate measured in the device.

FIG. 15 shows image processing for optical sweat rate measurement. Blue dye aids in identifying the channel length occupied by sweat. In AutoCAD, a trace is made of this filled blue length and of the microfluidic spiral diameter (dashed black), which has a known length (4.8 mm for the patch worn on the finger as shown below) that can be used to scale and convert the processing software trace length into real-world units. This length is multiplied by the cross sectional area A, (200 μm×70 μm) to give the total sweat volume in the channel. This volume is subtracted from the calculated volume at the subsequent time point and divided by the intervening time to calculate the average sweat rate between when the images were recorded.

FIG. 16 illustrates sweat rate measured on the thigh of a sedentary individual using two microfluidic patches that are placed adjacently.

FIG. 17 illustrates sweat rate measured on the forearm of a sedentary individual using two microfluidic patches that are placed adjacently with horizontal and vertical orientations respectively. Two microfluidic patches with 8-mm collection areas were placed near each other on the forearm, with one placed vertically and the other placed horizontally. Sweat rate was monitored for 1 hour while walking. The sensors show comparable results for the two orientations.

FIG. 18 shows a comparison of sweat rate measured by the microfluidic sweat patch and the gravimetric method. Sweat rate measured by the sweat patch is 2 times higher than that measured by the gravimetric method.

FIG. 19, panels a-c, shows calibration plots showing linear response of pH, Cl—, and levodopa sensors. Calibration curves of sensor signal versus analyte concentration for panel a) pH, panel b) Cl⁻, and panel c) levodopa. Data is obtained from the potentiometry and chronoamperometry curves in FIG. 6, panels f-h and demonstrates each sensor's linear response.

FIG. 20, panels a-i, shows the reproducibility, stability, and bending tests of pH, Cl⁻, and levodopa sensors. Panels (a)-(c) Reproducibility, panels (d)-(f) stability and drift analysis, and panels (g)-(i) bending tests using 0.66 cm radius of curvature for the pH, Cl—, and levodopa sensors.

FIG. 21, panels a-c, illustrates extended bending tests of pH (panel a), chloride (panel b), and levodopa (panel c) sensors. Sensor signals are reported before bending and after 200, 400, and 800 cycles of bending with 0.66 cm radius of curvature.

FIG. 22, panels a-b, illustrates the influence of (panel a) pH and (panel b) ionic strength of the solution on n=3 levodopa sensors. Note that a decrease in the sensitivity of 0.5×PBS is due to a slight decrease in pH of the buffer solution.

FIG. 23 illustrates levodopa sensor selectivity against common interferents in sweat, including uric acid, ascorbic acid, and glucose.

FIG. 24, panels a-b, shows the flow rate dependence test of Flow rate dependence test of (panel a) Levodopa and (panel b) pH sensors inside the microfluidic channel. Displayed numbers in the plot indicate flow rates in nL min⁻¹.

FIG. 25, panels a-b, illustrates one method of compensating for flow rate effects on the levodopa sensor signal. Off-body calibration of the levodopa sensor with no flow before on-body use is used to establish the sensor baseline. Using the on-body sweat rate measurement, the on-body levodopa signal can then be corrected using the flow rate dependence shown in a) (derived from FIG. 16 (panel a), to produce the compensated curve shown in panel b).

FIG. 26, panels a-b, shows the hydrogel influence of measured (panel a) Levodopa and (panel b) pH inside the microfluidic channel at a constant flow rate. Note that potential overshoot for the pH sensor is due to ion-exchange and temporary, local concentration differences as the membrane equilibrates with new solution. Further, upwards drift of the levodopa sensor upon changing the sample concentration arises due to equilibration as the higher concentration reaches and stabilizes at the sensor's enzyme layer.

FIG. 27 illustrates the results for a levodopa sensor tested in sweat as background fluid.

FIG. 28, panels a-c, shows an in situ sweat analysis to assist Parkinson's disease management. A healthy subject wore the microfluidic patch and had broad bean intake of (panel a) 1 dose=100 g and (panel b) 2 doses=200 g during the measurement duration. Panel c) The subject had spinach during the measurement duration as a control trial. Note that a patch containing 8 radial, interdigitated electrode spokes for sweat rate measurement was used in the trials in panel (a) and panel (c), while a patch with 24 radial electrode spokes was used in panel (b).

FIG. 29 illustrates heat generation analysis during on-body patch attachment. Thermal infrared images captured 5 min and 90 min into on-body sensor wear. There is negligible difference in local skin surface temperature produced by the patch.

FIG. 30 illustrates hydraulic pressure drop as a function of channel width.

FIG. 31 illustrates a comparison of smearing out of the concentration transition step at the sensor location (0.4 cm into the channel) at the three different secretion rates and extreme diffusivities.

FIG. 32 illustrates average sweat concentration in well.

### DETAILED DESCRIPTION

The difficulty of accessing naturally secreting sweat has limited the ability to explore and utilize its rich information for non-invasive health assessment in sedentary individuals without active sweat induction. To address this, we developed a wearable patch that provides natural sweat collection and continuous analysis at various body parts. By devising a small microfluidic device with a hydrophilic filler (e.g., a laminated hydrophilic filler) and sweat sensors, we enable continuous sweat collection and analysis at low secretion sites like wrist even when the subject is physically inactive. It is demonstrated herein that the patch can track sweat variations arising from light physical activities, metabolic changes due to insulin injection and drug administration to assist Parkinson's disease management.

Accordingly, in certain embodiments a wearable microfluidic device to measure natural sweat secretion rates and compositions is provide as well as uses thereof. The device presents an important advancement in wearable sweat sensing by allowing continuous perspiration analysis without artificial sweat induction in sedentary individuals. It enables local sweat rate measurements even at the lowest sweat secretion regions and allows investigation of relation between perspiration and physical and physiological functions. We overcome the challenge of accessing natural sweat through the use of a microfluidic device embedded with a hydrophilic filler (e.g., a laminated hydrophilic filler) inside a collection well. The filler minimizes the dead volume originated from the well and enhances sweat transport with minimal lag time.

In various embodiments the device dimensions were designed through consideration of the flow resistance in sweat glands and microfluidic channels according to the Poiseuille's law. By integration of an electrical sensor for sweat rate monitoring and electrochemical sensors for pH, Cl⁻, levodopa (or other analyte) detection, we enabled continuous analysis of natural sweat rate and composition. As described in Example 1, we utilized the device to measure natural sweat secretion rates on various locations of a human subject including shoulder, chest, bicep, wrist, abdomen, thigh, and leg, and finger. We also explored dynamic sweat behaviors during light physical activities, hypoglycemia, and control drug administration for Parkinson's disease management.

The device(s) described herein proves to be an ideal platform to continuously or routinely monitor users' medical conditions and physiological status during daily routines. They can also advance sweat investigations beyond what current wearable sweat sensors can provide by promoting a fundamental understanding of natural sweat secretion and its relation to diverse health conditions.

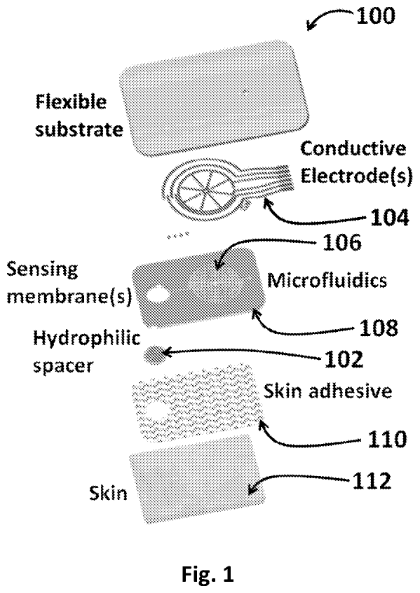

Components of one embodiment of a microfluidic sweat analysis patch designed to enable effective small volume collection and analysis of natural sweat are schematically illustrated in FIG. 1. As shown therein, the sweat analysis patch 100 includes three major components: a microfluidic layer 102, which in certain embodiments can comprise a microfluidic chip, electrochemical and electrical sweat sensing electrodes 104, and a hydrophilic filler 106. In certain embodiments the hydrophilic filler is laminated. As displayed in FIG. 1 the microfluidic layer 102 which, in certain embodiments, can comprise a polydimethylsiloxane (PDMS)-based microfluidic chip contains a collection well 108 and a microfluidic channel 110. In use, the collection well 108 interfaces with the skin 116 and its area can be varied/modulated to acquire varying amounts of sweat. The microfluidic channel 110 connects the collection well 108 to an outlet 118. As illustrated in FIG. 1, the microfluidic channel contains two intertwined spirals, although other configurations can readily be utilized.

In certain embodiments the microfluidic layer 102 (e.g., microfluidic chip) is aligned and bonded together with the sweat sensing electrodes 104 so that the sweat sensing electrodes are in contact with a fluid (e.g., sweat) in the microfluidic channel(s) 110. In various embodiments the sensing electrodes are configured for detection of sweat rate (e.g., as an impedance-based sweat rate detector. In certain embodiments the sensing electrodes are functionalized for detection of physiological analytes, e.g., pH, Cl—, levodopa and other drugs, and the like.

In the embodiment illustrated in FIG. 1 sensing electrodes contain four outer semi-circles surrounding two interdigitated wheel-shaped electrodes. The electrochemical sensors such as pH, Cl⁻, and levodopa are functionalized on the semi-circles, and the central interdigitated wheel acts as an impedance-based sweat rate sensor. It will be recognized, however that different sensor electrode configurations can be utilized, e.g., along with different microfluidic channel configurations.

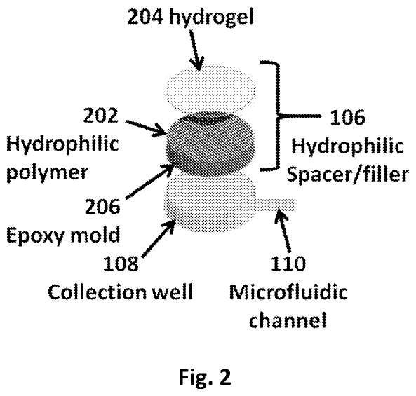

Finally, in various embodiments, the collection well 108 is filled with a patterned hydrophilic filler comprising for example an SU8 filler coated with a thin saturated hydrogel layer (see, e.g., FIG. 2). The patch can be worn on areas such as the finger and wrist without interrupting human activities.

Microfluidic Channel(s)

It will be noted that in various embodiments, the microfluidic channel 110 can be provided in any of a number of configurations and configured with a size and shape to optimize channel volume. In certain embodiments the microfluidic channel 110 comprises a serpentine/convoluted channel to increase channel length. In certain embodiments the microfluidic channel 110 comprises a circular spiral serpentine channel, an oval spiral serpentine channel, a square spiral serpentine channel, a switchback serpentine channel, a branched channel pattern, and the like. In certain embodiments the microfluidic channel can comprise a single channel or a plurality of microfluidic channels. As noted above, in the embodiment illustrated in FIG. 1, the microfluidic channel comprises two intertwined spirals.

With respect to the microfluidic channel 110 configuration and dimensions, it is noted that the natural sweat secretion rates of humans varies significantly with body location, For example, sweat secretion rates can be lower than 10 nL min⁻¹ cm⁻² at low secretion sites such as the arm or leg, and can reach on the order of 100 nL min⁻¹ cm⁻² at high secretion areas like the palm and foot. Such secretion rates, however, are small compared to typical sweat rates obtained by active sweat stimulation, which can be higher by an order of magnitude.

To enable low natural sweat rate measurement inside the microchannel, the channel cross-section needs to be as small as possible such that temporal variations in secretion rate can be resolved. However, the microchannel 110 length needs to be long enough to enable long-term measurement on desired body location. Accordingly, in certain embodiments, the microfluidic device is configured to contain ˜750 nL or greater such that sweat analysis can be done longer than an hour at the lowest sweat rate sites. Toward this goal, two constraints were considered:
(i) The sweat secretion process should not be critically impeded by the microfluidic dimensions; and
(ii) The flow rate in the microfluidic channel(s) 110 should not significantly be influenced by the viscous resistance along the channel length.

To meet these criteria, we examined the fluid resistance of the sweat glands and the microchannel based on the Poiseuille's law. In particular, as described in Example 1, the smallest dimensions enabled to reach the same fluid resistance as that of the sweat glands were computed.

Based on the calculations, a microfluidic channel cross-sectional area of 47 μm×47 μm satisfied the first constraint, and a channel length of 33 cm achieved a minimum volume of about 750 nL. However, this alone does not tell the flow rate variation along the channel length. Therefore, as described in Example 1, we next examined the flow rate variation due to the viscous resistance along the microchannel.

It was determined that flow rate decreases as sweat travels deeper into the channel, and the effect becomes more apparent as the channel width decreases and the length increases. As illustrated in Example 1, to contain a volume of ˜750 nL and a flow rate drop of less than 10%, it is desirable to have the cross-sectional area above 70 μm×70 μm and the microfluidic channel 110 length shorter than about 15 cm. These dimensions also satisfy the first constraint. Therefore, we chose two cross-sectional areas, 70 μm×70 μm and 200 μm×70 μm with lengths shorter than 15 cm to monitor sweat rates in low and high secretion regions respectively.

Hydrophilic Material.

It is ideally beneficial for microfluidic collection area (e.g., the area of the collection well 108 juxtaposed to the skin 118) to be large to maximize the accessible sweat glands. However, a large collection area creates a dead volume that must first be filled with sweat before the sweat flows into the microchannel. This creates a lag time in the sensor's response. To address this problem, we incorporated a hydrophilic filler to occupy the dead volume and to draw sweat readily into the channel as soon as it secretes. Accordingly, in certain embodiments the hydrophilic filler 106 comprises a hydrogel (e.g., an agarose-glycerol (AG-GLY) hydrogel 204) (see, e.g., FIG. 2). In certain embodiments a hydrogel was not used alone as a filler because it can dilute sweat compositions and hence put a challenge on the detection limit and sensitivity of electrochemical sensors. Accordingly, in certain embodiments, the hydrophilic material comprises a patterned mold 206, e.g., a patterned epoxy (e.g., SU8) coated with a hydrophilic polymer film 202. In certain embodiments the patterned mold 206 (e.g., SU8 filler) is patterned with grooves to enhance adhesion between hydrophilic film and SU8 (see, e.g., FIG. 2). The hydrophilic film contains two layers: a hydrophilic polymer 202 (e.g., polyvinyl alcohol (PVA)) and a hydrogel 204 (e.g., an agarose-glycerol (AG-GLY) film. In the embodiment illustrated in FIG. 2 hydrophilic polymer (e.g., the thin PVA film) covers the entire mold 206 (e.g., SU8 filler). A single PVA layer is brittle and can easily expose the hydrophobic pathway along the cracks. This will introduce pressure against sweat secretion due to surface tension and can prevent effective transport of sweat from the skin surface into the channel. By addition of the deformable hydrogel (e.g., AG-GLY gel) with high hydrophilicity, sweat from the collection area can be drawn into the gel and transported to the microchannel more effectively. Therefore, in certain embodiments, the hydrogel 104 (e.g., AG-GLY) film covers the top surface of the filler and, when the device is in use, is directly in contact with the skin. Without the hydrophilic filler, a collection well with a 5 mm diameter and a 400 μm thickness will require more than 2 hr to fill the well if sweat secretes at 300 nL min⁻¹ cm⁻². The integration of the hydrophilic filler enhances the collection and transports fluid into the channel within a few minutes. For a 5 mm diameter collection area, the film can hold a liquid volume of nearly 200 nL in the well. For a typical finger sweat rate (˜300 nL min⁻¹ cm⁻²), it takes approximately 3 mins to fill the well and initiate the sweat analysis. This is evident by the experimental observations and on-body experiments described in Example 1.

The structure and function of the hydrophilic insert 106 in the collection chamber 108 is also schematically illustrated in FIG. 12.

The time required for initiating sweat analysis using the device described in Example 1 is slightly longer than the theoretical calculations demonstrated for stimulated sweat in previously proposed device. However, with modification of the hydrophilic material and device design, it is possible to enhance the time required to initiate natural sweat analysis. Unlike prior devices that utilize hydrophilic material that has direct contact with the sensor and the skin for compositional analysis of stimulated sweat, the device described herein separates the hydrophilic filler from the sensing channel such that sensor will not be affected by the film and controls exact amount of fluid in the sensing channel for consistent sensor readings. Using the device, described herein detection of flow rate as low as 2 nL min⁻¹ was enabled.

Sensor Electrodes.

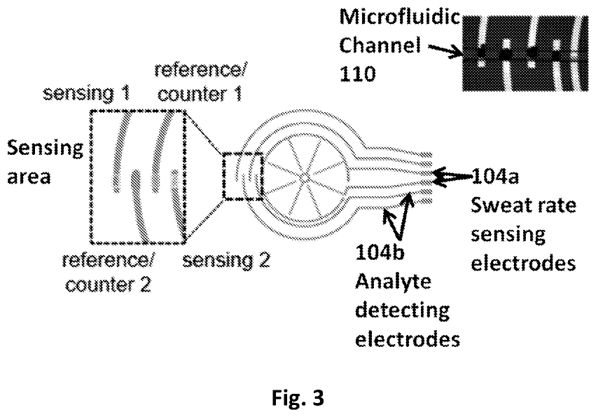

In order to utilize microfluidic, deice described herein for electrical measurement, electrical sensing electrodes 104 are incorporated into the device. In the illustrative, but non-limiting embodiment shown in FIG. 3, two interdigitated wheel shape electrodes 104a are aligned with the microfluidic channel and act as a sweat rate sensor. Each sweat rate sensing electrode consists of four radial conductive electrodes 104b1. As fluid is transported through the channel, it contacts an increasing area of the radial electrodes. With each contact by fluid, the impedance decreases because of a decrease in the resistance between the two electrodes, and a pulse indicating a change in admittance (inversely proportional to impedance) is observed. By counting the number of pulses and time interval between each pulse, the volume contained in the channel and sweat rate can be computed. A larger number of radial electrodes allows for higher temporal resolution of sweat rate measurements. Electrochemical sensors 104b1 located at the end of the semicircular electrodes are aligned with the microchannel as shown in FIG. 3. This allows electrochemical analysis as soon as sweat secretes into the channel. Depending on the sensing mechanism, either electrical current or potential is monitored.

The sweat rate sensor was first characterized by measuring admittance in different concentrations of NaCl solutions at an operating frequency of 100 kHz as described in Example 1. This frequency was chosen to minimize the capacitance contribution of the impedance and to maximize the resistive part of the impedance measurement. The relationship between admittance and fluid volume in the channel for NaCl concentrations of 10, 50, 100, and 200 mM was determined and it was demonstrated that, that at higher NaCl concentrations, the admittance between the electrodes increases due to the higher conductivity of increasing ion concentrations. Additionally, increasing fluid volume in the channel gives rise to higher admittance as more ionic solution is in contact with a larger area of the electrodes, decreasing the resistance between the electrodes. To demonstrate the reliability and reproducibility of the sweat rate sensors, it was also necessary to show that the time interval between admittance pulses are the same for a given flow rate in the channel and for fluid volume between the two contacts regardless of ions concentration. Using a commercial syringe pump, 10 and 200 mM NaCl solutions were flowed at a constant rate of 250 nL min⁻¹ into the sweat rate sensor and the volumetric increments between consecutive contacts is determined as a function of time. In comparing the 10 mM and 200 mM volumes it was observed that the pulses occur at the same time, indicating a reproducible calculation of sweat rate.

Lastly, to verify that our sweat rate device accurately returns the correct flow rate, the measured flow rate calculated from our sweat rate sensor was compared against the known input pump rate of a commercial syringe pump system. The syringe pump was used to flow 200 mM NaCl inside the microfluidic channel at an input rate of 150 nL min⁻¹ and 400 nL min⁻¹ as described in Example 1 and it was observed that the input pump rate is in agreement with the measured flow rate from the device.

Additionally, electrochemical sensors that have a sensing area of 200 μm by 200 μm each were characterized. In the configuration shown in FIG. 3 two electrodes serve as reference/counter electrode, and two electrodes are functionalized to detect target analytes. Detailed fabrication steps are outlined in Example 1. pH and Cl⁻ sensors operate by measuring the potential difference between the ion-selective electrode and the reference electrode. All sensors showed high sensitivity within the physiological range. For levodopa sensors, we additionally performed the influence of pH and ionic strength on the sensor's performance and showed that sensitivity of levodopa sensor decreases with decreasing pH and remains relatively stable for variation of ionic strength.

### Uses of Wearable Sweat Sensors.

The wearable devices described herein find utility in a wide variety of applications. In various embodiments they can readily be used to detect sweat rate and/or one or a plurality of analytes.

In certain embodiments the devices are used to provide natural perspiration analysis during light physical activities. By way of illustration, as described in Example 1, the microfluidic patch was first used to monitor sweat dynamics to demonstrate if sweat can track different physical activities of a sedentary subject while performing routine tasks. The patch was placed on the wrist of a healthy volunteer, along with a heart rate monitor. Heart rate and sweat rate were simultaneously monitored for 6 hours. Results in showed that wrist sweat rate closely tracks heart rate arisen from various physical activities such as taking a walk and performing lab work.

We additionally conducted on-body sweat analysis on the finger and the wrist of a volunteer subject. A collection well of 3 mm diameter was used on the finger while an 8 mm diameter was used on the wrist for sweat analyses. This allowed hour-long measurement on both finger and wrist based on measured flow rates. Similar to the previous study, sweat rate, in general, closely tracked heart rate variations. Sweat pH remained stable at 6.8 and 7.1 on the finger and wrist throughout the measurement period. Sweat Cl showed slight variation initially and stabilized around 22 and 40 mM on finger and wrist respectively.

Resolution of wrist sweat rate can be enhanced by increasing number of radial electrodes in sweat rate sensors as discussed previously. Under our experimental conditions, we consistently observed perspiration in short time intervals (in second for the finger and in minutes for the wrist) throughout the day. Due to its ability to closely track different activities, the devices described herein can be beneficial for sweat investigations associating with physical and mental stress-induced sweat.

In certain embodiments the devices described herein can be used for the detection and/or quantification of sweat secretion induced by metabolic changes. By way of non-limiting illustration, as described in Example 1, the patch was utilized to investigate hypoglycemia-induced sweat secretion. In diabetic patients, injection of insulin gives rise to hyperhidrosis due to hypoglycemia. They can also be vulnerable to irregular heartbeat, which can be life-threatening. Understanding sweating and heart complications in diabetic patients, hence, can facilitate diabetes management. Toward this aim, we performed simultaneous monitoring of heart rate, sweat rate, and interstitial fluid (ISF) glucose levels to explore heart and sweat complications during large glucose variation. A diabetic subject wore the microfluidic patch on the finger along with a pulse oximeter. The measurement was done without interrupting the routine insulin injection procedures of the diabetic patient. During the measurement duration, the subject was asked to remain sitting without vigorous movements. Blood glucose was measured right before the measurement began and after it ended. ISF glucose data was recorded via Dexcom G6 continuous glucose monitor. As described in the trials in Example 1, glucose was initially high when the measurement began, and the sweat rate remained relatively low between 0.5 and 1 μL min⁻¹ cm⁻². After insulin was injected, glucose started to decrease rapidly. In the meantime, an increase in sweat rate was observed. When glucose further decreased lower than 90 mg/dL there was a dramatic increase in sweat rate up to 5 μL min⁻¹ cm⁻². Heart rate remained relatively unchanged during low glucose level. Based on our results, significant decrease in glucose level is accompanied by a rise in sweat rate while no clear heart rate irregularity is observed and this is readily detecting using the devices described herein.

In various embodiments the devices described herein can be used to detect and/or quantify one or a plurality of analytes. Illustrative analytes include, but are not limited a metabolite, a drug, ethanol, a metal ion, and/or a salt.

By way of non-limiting illustration, as described in Example 1, the devices described herein were used for levodopa sensing, e.g., for Parkinson's disease management. Levodopa is a first-line drug for treating Parkinson's disease. It has been reported that long-term intermittent oral dosage of L-dopa causes fluctuation in plasma levodopa concentrations and leads to unpredictable responses such as motor fluctuations and dyskinesia; thus, continuous monitoring of L-dopa is important to circumvent such unforeseen responses.

Sweat has been reported to contain foreign drugs, including levodopa. Sweat is a promising non-invasive way to continuously monitor levodopa level inside the body. It may also facilitate finding an optimal dosage and interval that is personalized to each patient. Additionally, Parkinson's patients usually suffer from abnormal sweating. Hyperhidrosis occurs when the blood levodopa concentration is low, therefore, studying sweat behavior and monitoring levodopa concentration can assist management of Parkinson's disease.

As described in Example 1, we conducted on-body trials to study how sweat levodopa evolves within the body. A healthy subject was asked to consume 100 and 200 g intake of broad beans which contain levodopa to observe sweat levodopa relation to broad beans intake. In this study, boiled broad beans which were reported to contain approximately 0.6% levodopa were used. Levodopa sensors were calibrated in sweat as shown in Example 1 to ensure measurement accuracy. A sweat collection well of 3 mm diameter was used. It was observed that levodopa was detected in sweat approximately 20 mins after initial intake and its concentration peaked at 35 mins after intake. The peak concentration was measured to be approximately 13 μM when the subject had 1 dose of levodopa (1 dose of levodopa=100 g of broad beans).

In another experiment, the subject again consumed 200 g of broad beans, and levodopa was measured approximately 20 mins after initial intake. Its concentration peaked at 35 μM, 30 minutes after initial intake and slowly decreased. Additional trials showed similar results. We observed that levodopa concentration in sweat increases with increasing doses. When other foods with minimal levodopa is consumed, no significant signal is observed. This indicates that monitoring sweat levodopa is a promising way to keep track of blood levodopa to assist medication management of Parkinson's disease patients.

In conclusion, the devices described herein provide continuous analysis of naturally secreting sweat at diverse locations of sedentary individuals. Based on our studies, the device is ideal for monitoring natural sweat behavior while performing day-to-day indoor activities. Our study of sweat dynamics based on physical and physiological changes also shows its promising future sweat applications and clinical investigations related to passive perspiration. The devices described herein may actualize routine health and psychological assessment such as emotional contentment and development of infants, rehabilitation after stroke and recovery from combat stress through further sweat investigations. They may also help discover new sweat relations to physiological and medical conditions by gleaning insight into natural sweat profile of individuals

### EXAMPLES

The following examples are offered to illustrate, but not to limit the claimed invention.

### Example 1

### A Wearable Patch for Continuous Analysis of Thermoregulatory Sweat at Rest

The body naturally and continuously secretes sweat for thermoregulation during sedentary and routine activities at rates that can reflect underlying health conditions, including nerve damage, autonomic and metabolic disorders, and chronic stress. However, low secretion rates and evaporation pose challenges for collecting resting thermoregulatory sweat for non-invasive analysis of body physiology. Here we present wearable patches for continuous sweat monitoring at rest, using microfluidics to combat evaporation and enable selective monitoring of secretion rate. We integrate hydrophilic fillers for rapid sweat uptake into the sensing channel, reducing required sweat accumulation time towards real-time measurement. Along with sweat rate sensors, we integrate electrochemical sensors for pH, Cl—, and levodopa monitoring. We demonstrate patch functionality for dynamic sweat analysis related to routine activities, stress events, hypoglycemia-induced sweating, and Parkinson's disease. By enabling sweat analysis compatible with sedentary, routine, and daily activities, these patches enable continuous, autonomous monitoring of body physiology at rest.

### Results and Discussion.

Device Structure.

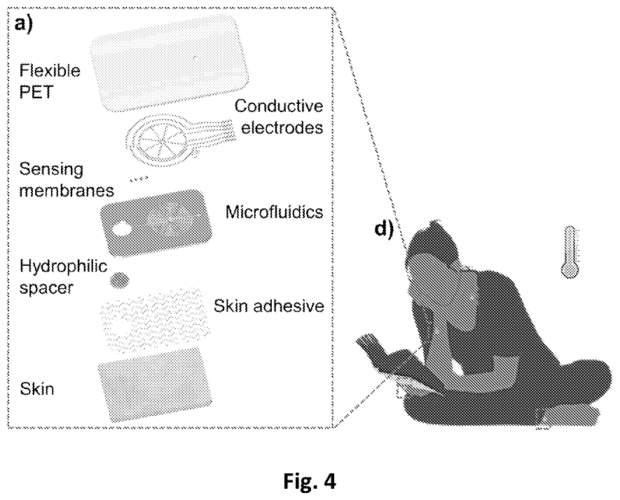

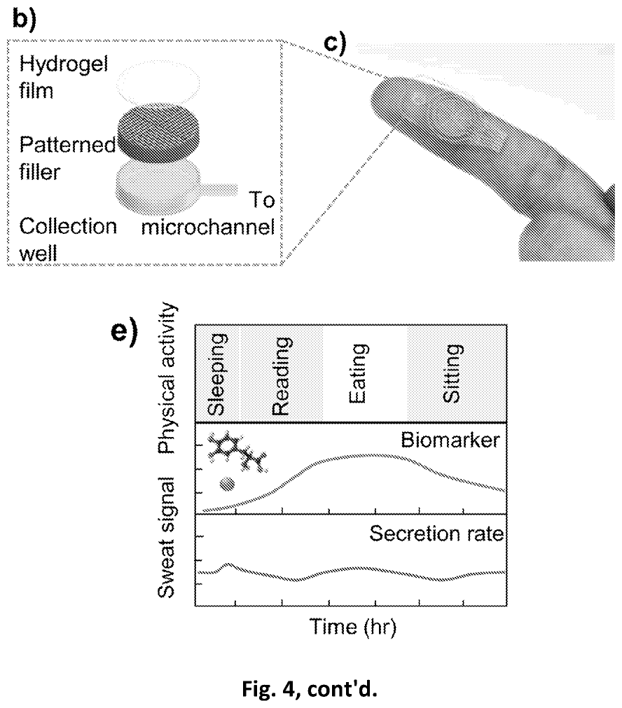

Our microfluidic device shown in FIG. 4 is designed to enable effective small volume collection and analysis of resting sweat. The device, as illustrated, includes three major components: a microfluidic layer, electrochemical and electrical sweat sensing electrodes, and a laminated hydrophilic filler. As displayed in FIG. 4, panel a, the polydimethylsiloxane (PDMS)-based microfluidic layer contains a collection well and a microfluidic channel. The collection well interfaces the skin and its area can be modulated to acquire varying amounts of sweat. The microfluidic channel contains two intertwined spirals, and the channel connects the collection well and the outlet. The microfluidic layer is aligned and bonded together with the sweat sensing electrodes. The sensing electrodes contain four outer semicircles surrounding two interdigitated wheel-shaped electrodes. The electrochemical sensors such as pH, Cl⁻, and levodopa are functionalized on the semicircles, and the central interdigitated wheel acts as an impedance-based sweat rate sensor. Finally, the collection well is filled with a patterned SU8 filler coated with a thin saturated hydrogel layer that contacts skin for sweat uptake (FIG. 4, panel b). The patch can be worn on areas such as the finger and wrist without interrupting human activities as pictured in FIG. 4, panels c, d.

Device Design.

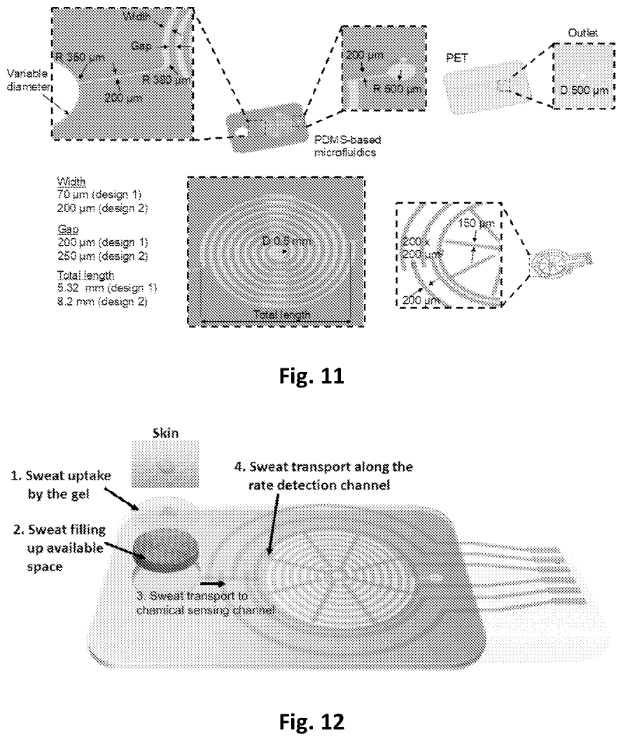

Humans' sweat secretion rates at rest vary across different body locations on average. For instance, sweat secretion rates can be lower than 10 nL min⁻¹ cm⁻² at low secretion sites such as arm and leg, and can reach on the order of 100 nL min⁻¹ cm⁻² at high secretion areas like the palm and foot^{29,30}. Such secretion rates are small compared to typical sweat rates obtained by active sweat stimulation, which can be higher by an order^{29,31}. To enable low resting sweat rate measurement inside the microchannel, the channel cross-section needs to be as small as possible such that temporal variations in secretion rate can be resolved by allowing fast speeds of the moving sweat front. At the same time, the channel resistance cannot be so high as to limit flow in the channel and potentially suffocate sweat gland secretion, so the channel cross section cannot be too narrow. Finally, the channel length needs to be long enough for the device to have sufficient volumetric holding capacity to enable long-term measurement on desired body locations. Here, we aim to develop a microfluidic device that can contain ˜750 nL or greater such that sweat analysis can be done longer than an hour at the lowest sweat rate regions. Toward this goal, we estimated secretory pressures of the sweat gland spanning a broad range of resting sweat secretion rates from 3 to 1 μL min⁻¹ cm⁻². We established that the channel contributes to most of the device hydraulic resistance compared to the collection well. For various square cross-sectional areas and associated channel lengths that give close to 750 nL holding capacity, we calculated hydraulic pressure losses and compared these to the secretory pressure of the grand. From this, we established that a channel cross section of 70 μm×70 μm with ˜15 cm length has low enough resistance to support sweat flow across low to high secretory rates. Detailed calculations of this procedure are reported on in the Supplementary Information. Based on these results, we chose two cross-sectional areas of the spiraling microfluidic portion for sweat rate measurement, 70×70 μm (design 1) and 200 μm×70 μm (design 2) as depicted in FIG. 11, with lengths shorter than 15 cm to monitor sweat rates in low and high secretion regions, respectively. Note that channels on the order of 10's of microns wide have been previously demonstrated for wicking nanoliters of sweat off the skin surface and onto the sensor²⁵. In contrast, the spiraling channel design used here is crucial not only for capturing low sweat volumes, but for efficiently drawing it over interdigitated electrode spokes for selective, continuous sweat rate measurement within a consolidated sensor footprint.

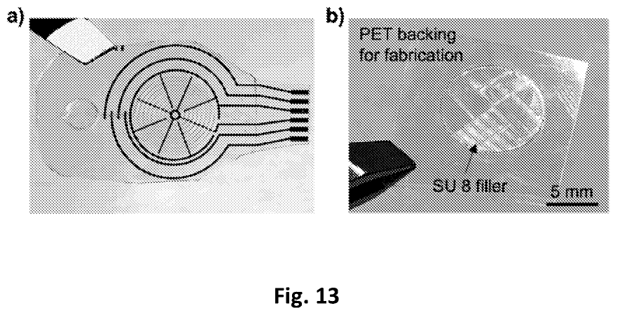

It is ideally beneficial for microfluidic collection area to be large to maximize the accessible sweat glands. However, a large collection area creates a dead volume, in which sweat firstly needs to be filled before flowing into the microchannel. This creates a lag time in sensor's response. To address this problem, we incorporated a hydrophilic filler, containing a patterned SU8 mold and hydrogels, to occupy the dead volume and to draw sweat readily into the channel as soon as it secretes. Hydrogels have been used extensively in the wearable electronics community to create soft interfaces and to absorb and hold biofluids onto sensor surfaces, but deploying gels to enhance sweat replacement times and minimize accumulation volumes and lag times represents a key advantage in this work³²⁻³⁴. This structure overall comprises of a PVA-coated rigid SU8 component that is first inserted into the well and overlayed with an agarose-glycerol hydrogel that directly contacts skin for sweat uptake (FIG. 12). Optical images of the filler and the assembled microfluidic are shown in FIG. 13. We did not use hydrogel alone as a filler because it can dilute sweat compositions and hence put a challenge on the detection limit and sensitivity of electrochemical sensors, so we instead use only a thin hydrogel layer and occupy the remaining dead space with the rigid filler. The ameliorating effects of this combination on mixing and analyte dispersion are detailed in the Supplementary Information, below. The filler further contributes to mechanical integrity, inhibiting collapse of the collection well under pressures which could otherwise artificially force fluid into the channel and create artefacts in measured sweat rate. The SU8 filler is patterned with grooves to alternate closed-off regions that diminish well volume with open regions roughly 100 μm-wide that allow sweat to pass through and into the device. The hydrophilic film contains two layers: a polyvinyl alcohol (PVA) and an agarose-glycerol (AG-GLY) film. The thin PVA film covers the entire SU8 filler. A single PVA layer is brittle and can easily expose the hydrophobic pathway along the cracks. This will introduce pressure against sweat secretion due to surface tension and can prevent effective transport of sweat from the skin surface into the channel.

By addition of the deformable AG-GLY gel³⁵ with high hydrophilicity, sweat from the collection area can be drawn into the gel and transported to the microchannel more effectively. Therefore, the AG-GLY film covers the top surface of the filler and is directly in contact with the skin. Without the hydrophilic filler, volumetric calculations show that a collection well with a 5 mm diameter and a 400 μm thickness will require more than 2 h to fill the well if sweat secretes at 300 nL min⁻¹ cm⁻² while taking over 30 min and 200 h for extreme rates of 1 μL min⁻¹ cm⁻² and 3 nL min⁻¹ cm⁻², respectively. The integration of the hydrophilic filler enhances the collection and transports fluid into the channel within a few minutes. For a 5 mm diameter collection area, the film can hold a liquid volume of nearly 200 nL in the well. For 300 nL min⁻¹ cm⁻², it takes approximately 3 min to fill the well and initiate the sweat analysis. Similarly, it takes under a minute for a rate of 1 μL min⁻¹ cm⁻² near the upper range of resting sweat secretion or around 30 min for rates toward the 3 nL min⁻¹ cm⁻² lower end when appropriately sized collection wells are used. The experimental result using a syringe pump supports this conclusion as shown in the supplementary materials. For typical resting sweat rates ˜<30 nL min⁻¹ cm^{-2 29}, the difference in lag time is more apparent (˜30 min instead of ˜ a day), and sweat measurement is almost impractical for a hollow PDMS well. Note that due to its small footprint and the fact that the sensing patch is held tightly against skin via medical adhesives, the hydrogel cannot swell so much that it pushes off from the skin surface and delaminates the patch. Instead, as the hydrogel uptakes sweat, the tight seal against skin forces the hydrogel to expel this sweat into the channel. This supports rapid and leakproof collection of resting sweat in the channel. With further investigation of the hydrophilic film and device design, it is possible to enhance the time required to initiate thermoregulatory sweat analysis at rest. Unlike prior devices which utilize hydrophilic material that has direct contact with the sensor and the skin for compositional analysis of stimulated sweat^{22,25,36}, our device separates the hydrophilic filler from the sensing channel such that the sensor surface is not impacted by fluid and pressure variations in the film, and to control and fix the amount of fluid in the sensing channel for consistent sensor signals. Using the device, we also enable detection of flow rate as low as 2 nL min⁻¹ as presented in FIG. 14, panel b.

Due to low resting sweating rates and the dimensions of the well and channel, we expect some diffusion and Taylor dispersion of analyte concentrations between when sweat is secreted on the skin surface and when it arrives at the electrochemical sensors near the entry of the channel. We perform a careful study of the time lags associated with this spread of analyte profiles in the Supplementary Information. Regions like the fingertips and hands are established to have relatively higher resting sweating rates, for which our simulations indicate a time lag of around 3 min³⁰. This lag presents a limit on how updated the continuously made measurements are, but is well below the time scale over which physiological changes are expected to be manifested in sweat. At lower rates, sweat intrinsically moves more slowly through the device and takes longer to arrive at the sensors, allowing more time for dispersion effects. In contrast, because sweat rate is measured simply by the rate of fluid front movement, continuous and updated sweat rate measurements can be made with negligible time lag once sweat enters the channel.

Device Feasibility for Sweat Collection at Rest.

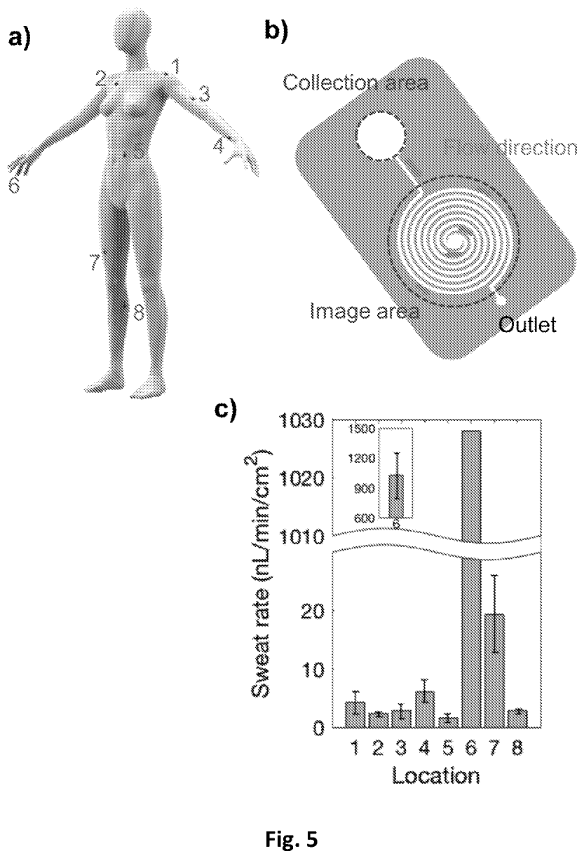

It is important to explore at-rest thermoregulatory sweat secretion rate as it is modulated not only by environmental conditions and physical activities but also by mental stimulation and underlying health conditions^{7,8,37-40}. Tracking sweat secretion routinely may help discover valuable insights into human physiology (FIG. 4, panel e). Toward this goal, we first tested the feasibility of our microfluidic collector. We performed on-body sweat collection on various body sites, including shoulder, chest, bicep, wrist, abdomen, finger, thigh, and leg as displayed in FIG. 5, panel a. The patches were worn by a volunteer individual (subject 1) for 24 h, and optical images were taken periodically. Patches with different collection areas were used to capture sweat rate in a practical time frame; specifically, smaller collection area was used in high secreting regions like the fingers while larger areas were used in lower secreting regions like the chest as described in Table 1.

TABLE 1
Typical sweating rates and appropriate patch collection
areas to enable sweat rate measurement over practical
time scales at different body sites.

| | Typical sweat rate | Collection diameter |
|---|---|---|
| Location | (nL min⁻¹ cm⁻²) ²⁹ | (mm) |
| Chest | 10-40 | 10 |
| Upper arm | 10-150 | 5, 10 |
| Forearm | 10-30 | 10 |
| Abdomen | 10-40 | 10 |
| Finger | 60-200 | 3, 5 |
| Leg | 10-40 | 10 |

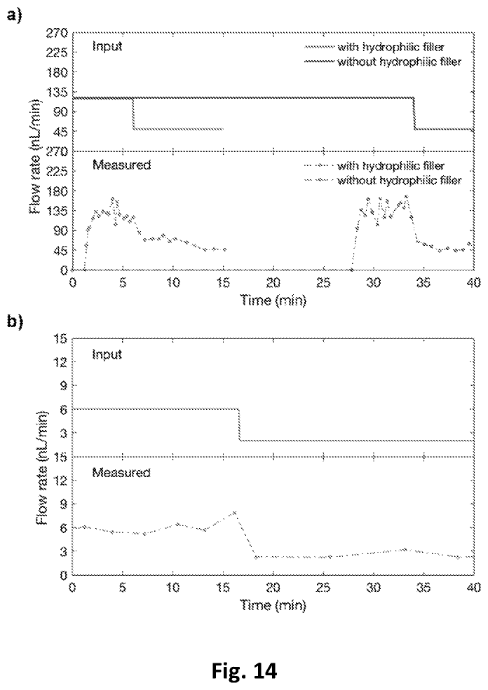

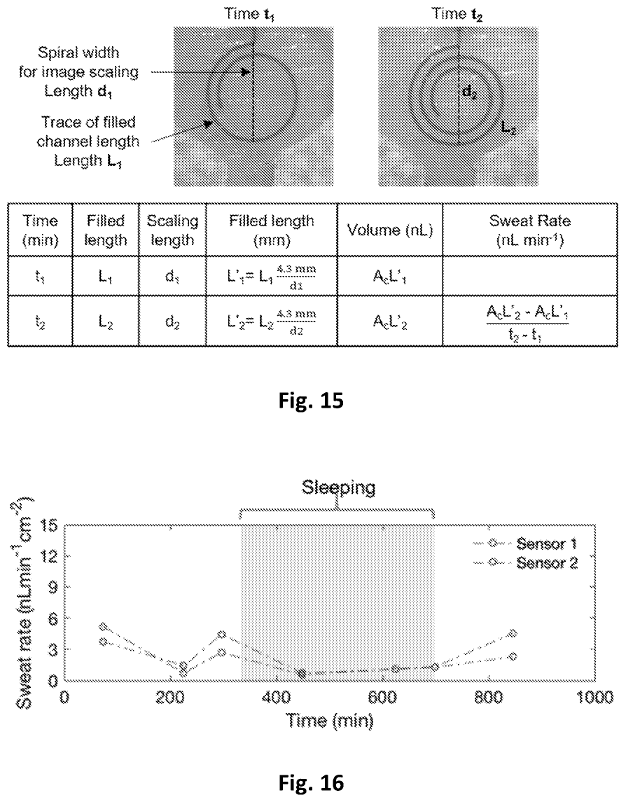

The subject was asked to refrain from moderate to vigorous physical activities during the 24-h time frame. An example of the collection area and the imaging area of the patches are displayed in FIG. 5, panel b. Depending on the targeted regions, the patches differed in collection area and microfluidic dimensions. Color dye was used in the hydrogel to ensure sweat flow in the channel could be clearly observed. The first images in each location were taken as soon as sweat secretion began in the image area. It took 2-60 min to begin collection depending on targeted locations. FIG. 5, panel c shows a bar chart of average sweat rates on the eight locations. These values are obtained optically based on FIG. 5, panel d, with FIG. 15 detailing the method of calculating sweat rate from images of dyed sweat progression in the channel. Further, the patches were put on three additional subjects to measure sweat secretion rates at various locations. Measured sweat rate averages are displayed in Table 2.

TABLE 2
Measured average sweat rate of three subjects on different
locations. Three patches are placed per subject, with
“—” indicating a bare site without a sensor attached. Displayed
sweat rates are in unit of nL min⁻¹ cm⁻².

| Location | Subject 2 | Subject 3 | Subject 4 |
|---|---|---|---|
| Bicep | — | 6 ± 3.3 | 9 ± 1.2 |
| Wrist | 5.5 ± 1.3 | — | 7.4 ± 3.7 |
| Finger | 300 ± 93.5 | 101 ± 34 | 620 ± 202 |
| Leg | 3.5 ± 1.4 | 2.4 ± 1.2 | — |

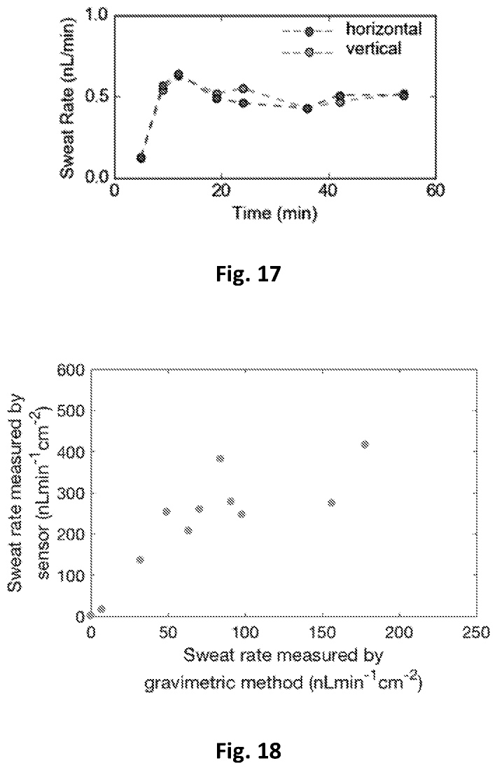

According to our results, the finger has the highest secretion rate that can range between the order of 0.1 and 1 μL min⁻¹ cm⁻². All other regions show relatively low secretion rate of 1-20 nL min⁻¹ cm⁻². The results agree with the literatures which showed that palm and fingers have the highest secretion rate²⁹. Majority of our measured sweat rates are slightly lower than reported rates in literatures possibly due to lower environmental temperatures and humidity used in the experiments. To demonstrate the reproducibility of the sweat rate measured by the patch, we also conducted a trial where we had a subject wearing the patches on two adjacent locations on the thigh. The data is displayed in FIG. 16. The two patches show similar sweat rate trends and nearly identical rates when the subject was sleeping. In addition, FIG. 17 compares sweat rate measurement from two patches placed near each other on the forearm, with one oriented horizontally and the other vertically. The orientation does not greatly impact sweat uptake into the device. Finally, note that the when the patch is worn on a finger, it can extend across the upper finger joint (as seen in FIG. 4, panel c) and inhibit bending. While this is not highly disruptive as a relaxed finger is naturally relatively straight at the upper joint, more compliant substrates can be used in future to ensure reliable sweat uptake even with significant bending across covered joints.

There are a few factors that may induce uncertainty in measured sweat rates values. They include possible sweat migration into the collection area from other parts underneath the patch. In addition, there is a possibility of higher sweat rate in the collection area to make up for the perspiration that may be hampered in the rest part of the device. These factors can result in overestimation of the measured sweat rates; however, the relative sweat rates will not differ. To investigate the first concern, we spot colored dye on the underside of the patch. After device removal, we observe that skin is dyed just in the region of the collection well and not in surrounding regions, confirming that there is no lateral sweat leakage or transfer from the collection well, and all sweat produced in that area is forced into the device for measurement. The dyed sweat can be visually monitored as it flows in the channel to optically validate electrical sweat rate measurements or as an independent visual measurement scheme enabled by this patch. This scheme for optical sweat rate tracking is realized via discrete photographs of sweat progression within the channel as in FIG. 5.

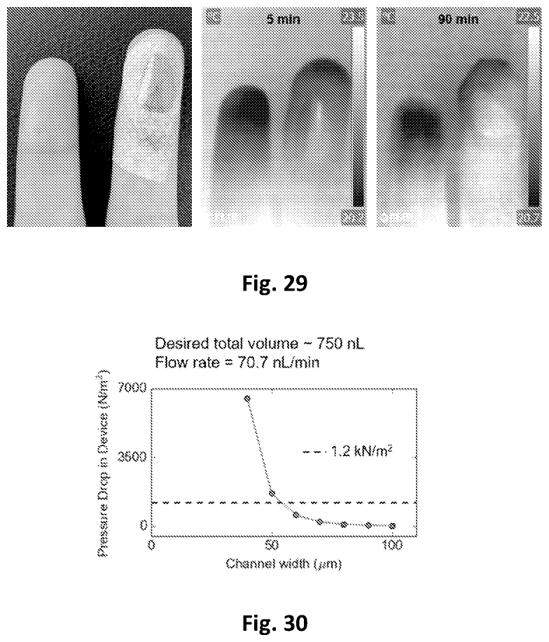

As for the second factor that could impact sweat rate accuracy, namely compensatory sweating effects, all devices covering sweat glands can induce the same effect, and this requires careful studies in the future. Local heat generation due to on-body attachment of the patch must also be considered as it could potentially elevate sweating rates18, but negligible local heating is observed as demonstrated in FIG. 29 due to the small patch size and at or near rest conditions. We compared the measured sweat rates from the patches with more traditional gravimetric analysis. For the latter, an absorbent pad is held against skin for sweat accumulation and weighed before and after each sweat collection that lasts approximately 20-30 min. The pad is placed in a shallow 0.5 cm2 chamber to minimize evaporation during sweat collection. A new patch was used in each sweat collection. The patch and the pad were placed on ring and pinky fingers to simultaneously collect sweat. Results are displayed in FIG. 18, which shows that the patch collects ˜2 times larger sweat amount per unit area than the pad. It is important to note that evaporation of the absorbent pad during removal from the skin surface and weighing can have significant effect on the measured amount of sweat. We discovered that the evaporation rate from the pad can be 200-400 nL min⁻¹ cm⁻², which is the same order of measured sweat rates. Hence, gravimetric measurement error can be on the order of 100%. This can lead to a lower sweat rate measured by the gravimetric method. This shows a key advantage of our device as it minimizes the uncertainty arisen from the evaporation. When dealing with low volumes and rates associated with at-rest sweat, our device encapsulates sweat immediately and uses a narrow channel to create rapid movement of the sweat front, translating into frequent and updated sweat rate measurements that overcome the evaporation and errors of gravimetric analysis. With these considerations, it is reasonable to assume that sweat under the collection area faithfully contributes to the measured sweat rate from the patch.

Sensors Characterization.

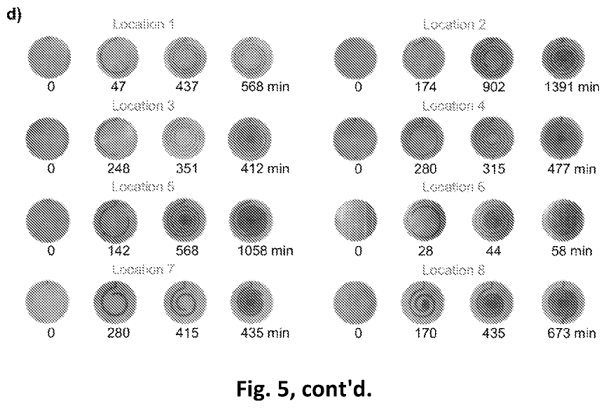

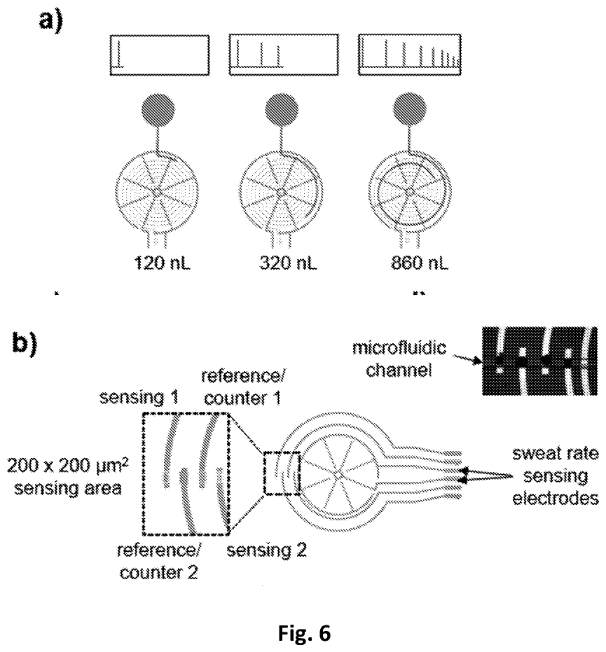

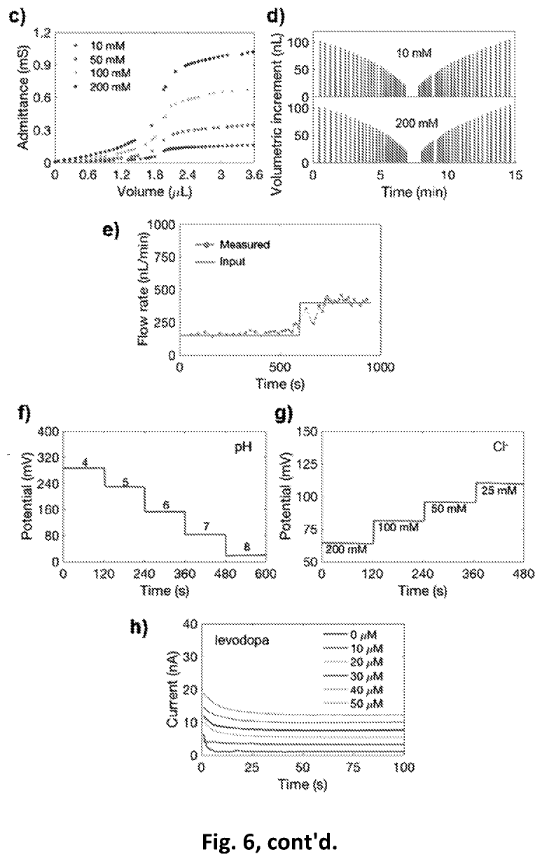

In order to utilize the microfluidic patch for electrical measurement, electrical sensing electrodes are incorporated into the microfluidic. As shown in FIG. 6, panel a, two interdigitated wheel shape electrodes are aligned with the microfluidic and act as a sweat rate sensor. The electrodes contain a total of 8-24 radial electrodes. At the initial contact, a sudden change in admittance indicates fluid entering the channel. As fluid is transported through the channel, it contacts an increasing area of the radial electrodes. With each contact by fluid, the impedance decreases because of a decrease in the resistance between the two electrodes, and a pulse indicating a change in admittance (inversely proportional to impedance) is observed. By counting the number of pulses and time interval between each pulse, the volume contained in the channel and sweat rate can be computed. In other words, as the spacing between the spokes is known and the channel cross-section is fixed, each time the sensor signal undergoes a discrete step change we can know how much additional volume of fluid was added to the channel. This allows an estimate of volumetric increment versus time, where the time points correspond to the time of the admittance step changes, as shown schematically by the signals in FIG. 6, panel a. Note that the spacing between spokes decreases as the channel spirals inwards and increases once it starts spiraling outwards. This causes the volume increment to decrease as the fluid front moves toward the center of the spiral, and to increase as it continues to move outwards.

A larger number of radial electrodes allows for higher temporal resolution of sweat rate measurements. Electrochemical sensors located at the end of the semicircular electrodes are aligned with the microchannel as shown in FIG. 6, panel b. This allows electrochemical analysis as soon as sweat secretes into the channel. Depending on the sensing mechanism, either electrical current or potential is monitored.

The sweat rate sensor (200 μm×70 μm) was first characterized by measuring admittance in different concentrations of NaCl solutions at an operating frequency of 100 kHz. This frequency was chosen to minimize the capacitance contribution of the impedance and to maximize the resistive part of the impedance measurement. FIG. 6, panel c demonstrates the relationship between admittance and fluid volume in the channel for NaCl concentrations of 10, 50, 100, and 200 mM. It can be seen that at higher NaCl concentrations, the admittance between the electrodes increases due to the higher conductivity of increasing ion concentrations. In addition, increasing fluid volume in the channel gives rise to higher admittance as more ionic solution is in contact with a larger area of the electrodes, decreasing the resistance between the electrodes. To demonstrate the reliability and reproducibility of the sweat rate sensors, it is also necessary to show that the time interval between admittance pulses are the same for a given flow rate in the channel and for fluid volume between the two contacts regardless of ions concentration. Using a commercial syringe pump, 10 and 200 mM NaCl solutions were flowed at a constant rate of 250 nL min⁻¹ into the sweat rate sensor. The volumetric increments between consecutive contacts is plotted as a function of time in FIG. 6, panel d. In comparing the 10 and 200 mM plots, it can be seen that the pulses occur at the same time, indicating a reproducible calculation of sweat rate. It is also important to note that the time interval spacing between each pulse is not the same for a constant flow rate in the channel because the fluid volume between consecutive contacts decreases as fluid travels toward the center and increases as fluid travels outwards from the center toward the outlet. For a 24-electrodes with 200 μm×70 μm channel, the time resolution is between ˜4 and 20 s for 50 nL min⁻¹ and can reach 2-9 min for 2 nL min⁻¹. For a 24-electrodes with 70 μm×70 μm channel design, the resolution is further enhanced. Lastly, to verify that our sweat rate device accurately returns the correct flow rate, the measured flow rate calculated from our sweat rate sensor was compared against the known input pump rate of a commercial syringe pump system. The syringe pump was used to flow 200 mM NaCl inside the microfluidic channel at an input rate of 150 and 400 nL min⁻¹ as shown in FIG. 6, panel e. It can be seen that the input pump rate is in agreement with the measured flow rate from the device, which is also evident in FIG. 14, panel b for lower flow rates. Note that an injection pump is used to conduct this benchtop analysis, and variation in how smoothly and consistently the pump injects at the preset rate causes fluctuations in the measured signal. This can be treated and potentially filtered as noise.

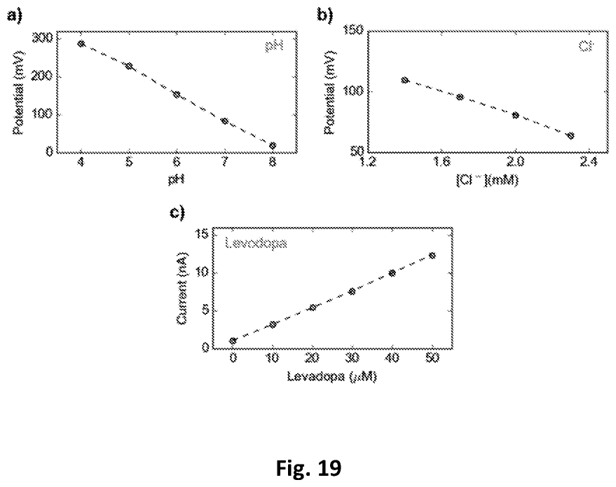

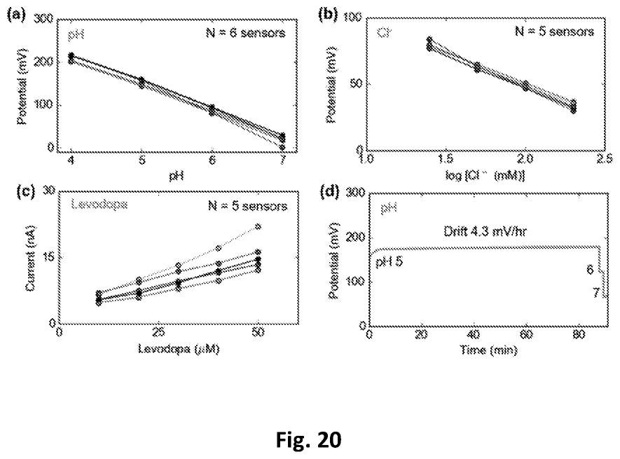

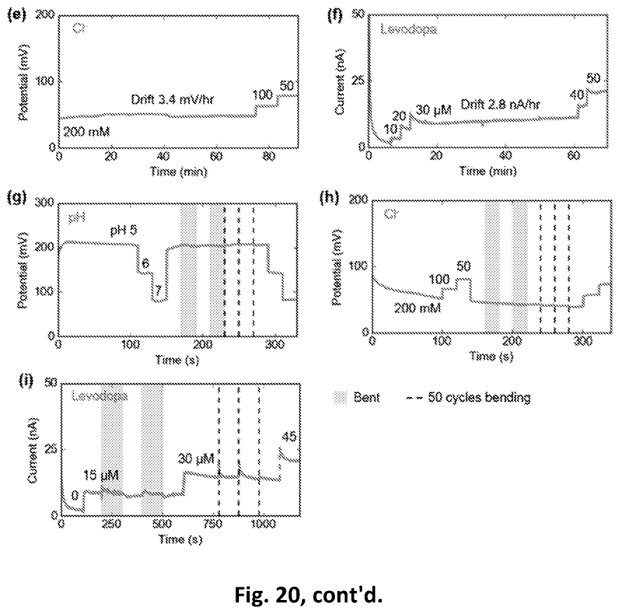

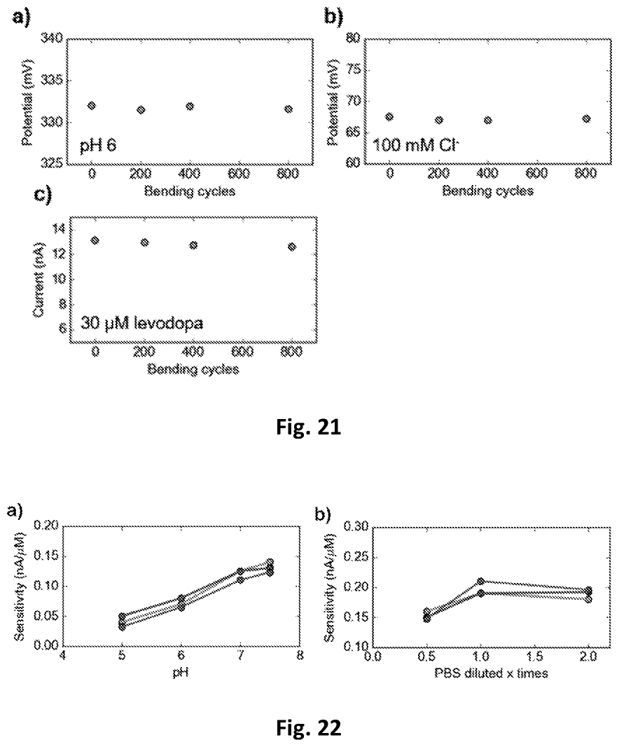

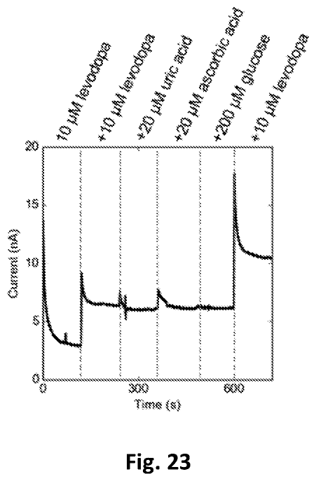

We further characterized the electrochemical sensors which have a sensing area of 200 μm by 200 μm each, given the 200 μm width of the functionalized electrode tips and the 200 μm width of the microfluidic channel in between the collection well and spiraling portion (as depicted in FIG. 11). As shown in FIG. 6, panel b, two electrodes serve as reference/counter electrode, and two electrodes are functionalized to detect target analytes. Detailed fabrication steps are outlined in the “Methods”. pH and Cl⁻ sensors operate by measuring the potential difference between the ion-selective electrode (ISE) and the reference electrode. The potential of the pH ISE changes with pH due to deprotonation of the ISE's conductive polyaniline film by H^{+ 27}. In contrast, the Cl⁻ ISE comprises of an Ag/AgCl electrode. A change in Cl⁻ concentration shifts the redox equilibrium between Ag and AgCl to create a measurable change in the sensor's potential signal. FIG. 6, panel f, g shows the performance of a pH sensor in pH 4-8 McIlvaine's buffer and a Cl⁻ sensor in solution containing 25-200 mM NaCl. Their sensitivities are measured to be 60 mV/pH and 55 mV/decade, which are close to Nernstian behavior. FIG. 6, panel h presents the performance of a levodopa sensor with a sensitivity of 0.2 nA μm⁻¹, an improvement in sensitivity per area compared to our previous work²⁸ due to an increased active surface area arising from modified fabrication detailed in the “Methods” section. The sensor measurement is based on current generated by enzymatic reaction between levodopa and tyrosinase. A small voltage applied to the levodopa sensor drives oxidation of levodopa by the tyrosinase enzyme, producing a Faradaic current that can be calibrated into a measure of levodopa concentration²⁸. All sensors show high sensitivity within the physiological range, with linear calibration curves shown in FIG. 19 as well as reproducibility, stability, and bending tests shown in FIG. 20 and FIG. 21^{14,27,28}. For the levodopa sensor, decreasing the sensing area has been a challenge as signal to noise ratio becomes significant. To address this, we optimized the sensing membrane with a thin conformal layer of mediator, an enzyme-immobilized layer, and a hydrophobic micellar membrane. Our sensor shows 2.5× enhanced sensitivity per unit area compared to the previously developed sensor²⁸ despite its smaller detection area, and has a response time under 20 s. Based on noise and drift, the sensor is expected to be able to discern down to 3 μM as non-negligible levodopa concentrations. We additionally performed the influence of pH and ionic strength on the levodopa sensor's performance. FIG. 22 shows that sensitivity of levodopa sensor decreases with decreasing pH and remains relatively stable for variation of ionic strength. Selectivity of this modified levodopa sensor is shown in FIG. 23.

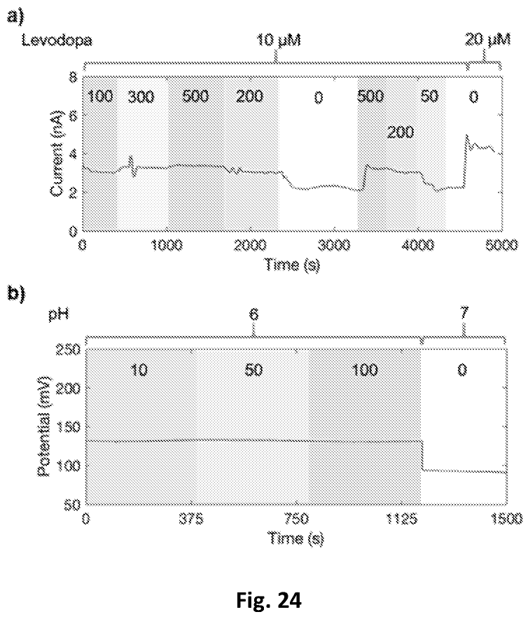

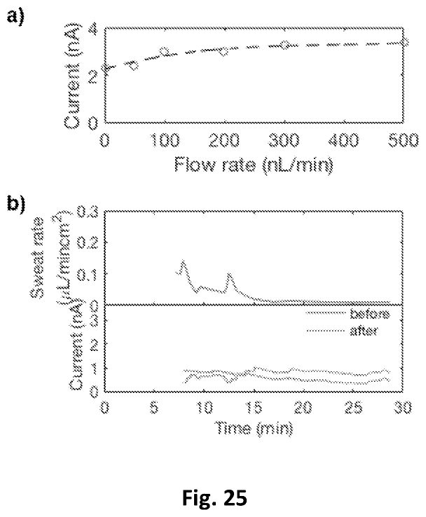

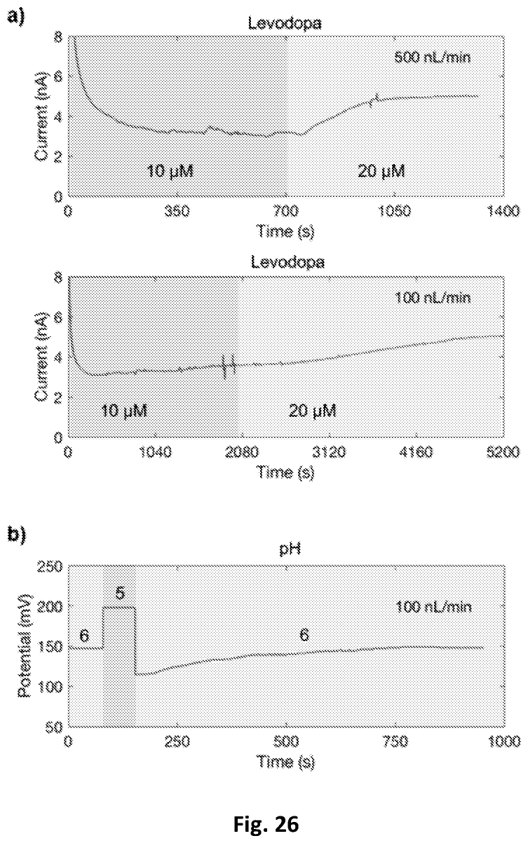

To further investigate the flow effect on the sensors' performances upon integration into the microfluidic channel, we performed flow dependence test as shown in FIG. 24. The levodopa sensor signal shows influence from flow rate that can be understood as follows: at lower rates, levodopa concentration is mass transfer limited and changes in flow rate more significantly impact the levodopa availability at the sensor surface. Above these rates (toward 100 nL/min and beyond), mass transfer of levodopa to the sensor surface is abundant and the sensor remains at a stable, higher signal level than at lower rates. The levodopa sensor (FIGS. 18, panel a and 25) shows an increase of approximately 0.02 nA for a change in flow rate of 10 nL min⁻¹. This dependence was considered when we computed the concentration of levodopa during on-body trials. It is important to note that, for on-body levodopa sensing, the sweat rate variations were generally <30 nL min⁻¹. FIG. 24, panel b shows a pH sensor as a representative of ion sensors, and the result indicates that pH sensor is not influenced by the change in flow rate. This is likely because H⁺ ions are small; hence, they quickly dope and de-dope with the polyaniline layer without limitation on mass transport. Further, the polyaniline layer is directly accessible to target ions in solution, whereas the enzyme of the levodopa sensor is covered by protective Nafion. This causes mass transfer limitations and flow rate dependence for the levodopa sensor, but not for the pH sensor. We further conducted experiments to investigate the influence of hydrogel on capturing true concentration of injected fluid. FIG. 26, panel a shows levodopa sensor that is initially loaded with 10 μm levodopa, and 20 μm levodopa solution was injected at a constant rate of 500 and 100 nL min⁻¹. For these flow rates, the sensor took less than 3 and 15 min, respectively to start responding to a change in concentration. To eventually reach the newly injected concentration, the sensor required about 6 and 40 min, respectively. On the other hand, the pH sensor (FIG. 26, panel b) took about a minute to detect change in pH and 10 min to replace the detection chamber with newly injected pH for an injection rate of 100 nL min⁻¹.

Near-Rest Perspiration Analysis During Light Physical Activities.

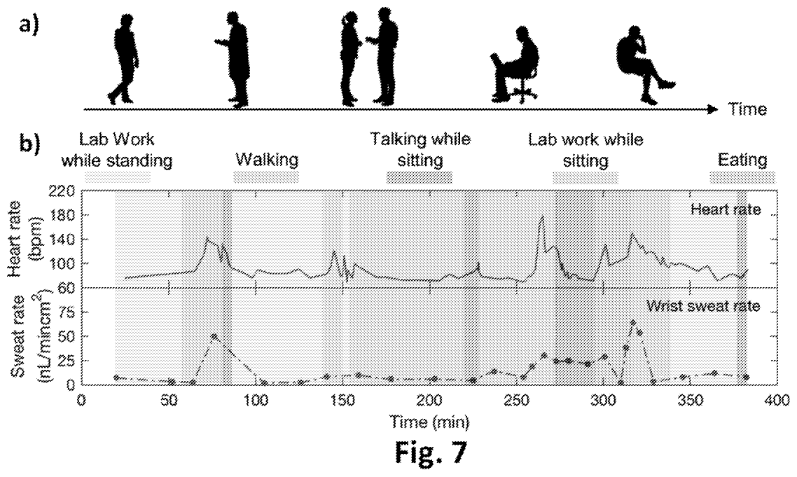

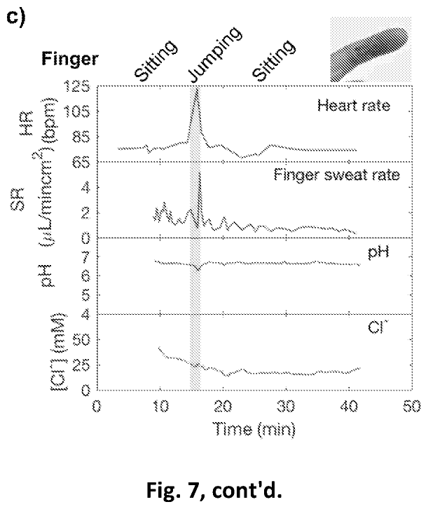

The microfluidic patch was first used to monitor sweat dynamics to demonstrate if sweat can track different physical activities of a sedentary subject while performing routine tasks (FIG. 7, panel a). The patch was placed on the wrist of a healthy volunteer, along with a heart rate monitor. Heart rate and sweat rate were simultaneously monitored for 6 h, with simple optical readout of sweat rate used over this extended sensing duration for convenience via the scheme detailed in FIG. 15. Results in FIG. 7, panel b, show that wrist sweat rate generally tracks heart rate stemming from various physical activities such as taking a walk and performing lab work. Specifically, sweating rates remained relatively low along with heart rate during more sedentary periods, while intervals of walking and other activities caused both to rise and subsequently fall. We additionally conducted on-body sweat analysis on the anger and the wrist of a volunteer subject using electrical sweat rate measurement. A collection well of 3 mm diameter was used on the anger while an 8 mm diameter was used on the wrist for sweat analyses, with the larger collection area on the list accounting for the lower expected rates of secretion at this site. This allows hour-long measurement on both anger and wrist based on now rates measured in FIG. 5, panel c and Table 2. To ensure microfluidic patches closely reflect actual sweat concentrations with a stable signal, we began analyses 10 min and 4 h after sweat secreted into the sensing channel. The time scales were chosen based on the subjects' average sweat rate shown in Table 2 and to achieve a stable sensing signal. For instance, for an average sweat rate of 300 nL min⁻¹ cm⁻², it takes ˜3 min for sweat to now into the sensing channel. To ensure we can capture a stable signal we waited until 10 min to initiate the measurement. FIG. 7, panels c & d shows finger and wrist sweat analyses as well as heart rate measurement on a healthy subject. Similar to the previous study, sweat rate, in general, follows changes in heart rate by elevating due to periods of activity and then restoring to lower levels. Sweat pH remained stable at 6.8 and 7.1 on the anger and wrist throughout the measurement period. Sweat Cl⁻ showed slight variation initially and stabilized around 22 and 40 mM on anger and wrist, respectively. This observation is supported by the literature⁴¹. Finger sweat rate showed higher resolution due to faster sweat secretion rate. Resolution of wrist sweat rate can be enhanced by increasing number of radial electrodes in sweat rate sensors as discussed previously. Under our experimental conditions, we consistently observed perspiration in short time intervals (in second for the anger and in minutes for the wrist) throughout the day. Due to its ability to closely track different activities, it can be beneficial for sweat investigations associating with physical and mental stress-induced sweat.

Sweat Analysis to Detect Stress Events Over 24 h.

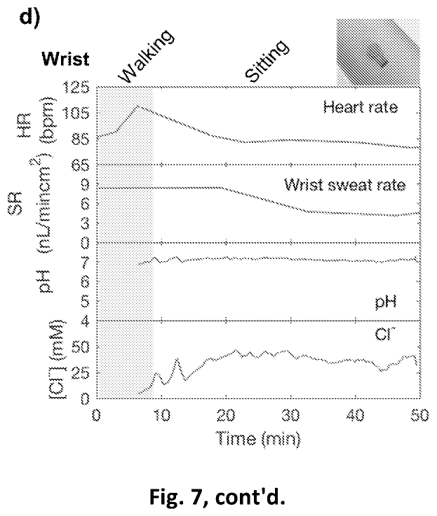

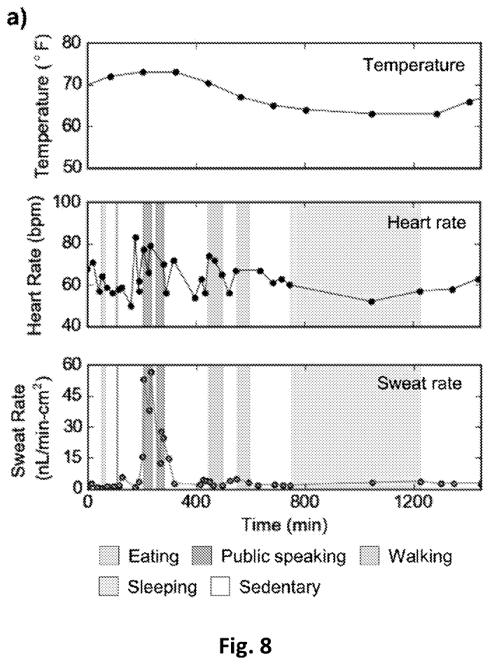

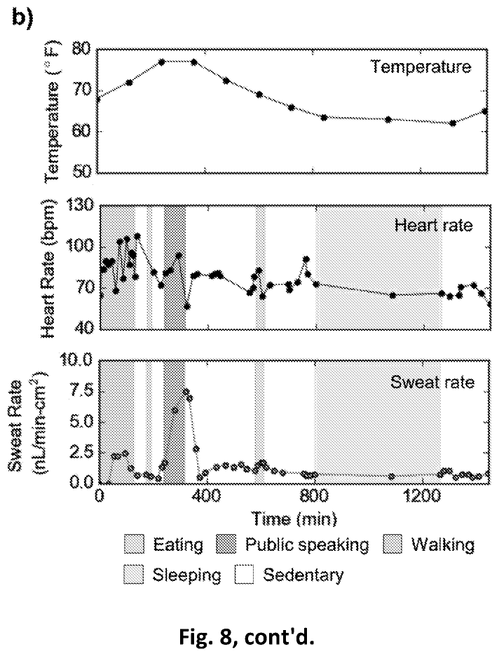

The patch was next worn on the fingertip of a healthy volunteer during two trials, 24 h each, with routine activity including eating, walking, and sleeping, while heart rate and ambient temperature were monitored simultaneously. The subject was mostly sedentary and performed intervals of public speaking including giving a presentation and answering questions in a live streamed conference in Trial 1 (FIG. 8, panel a), and teaching a class in Trial 2 (FIG. 8, panel b). These events generated a stress response in the body due to a combination of anticipation and public speaking that is reminiscent of the clinical standard Trier Social Stress Test⁴². Heart rate generally elevated in anticipation of and during the stress events in both trials, increasing a total of 28 bpm for the presentation in Trial 1 and 21 bpm while teaching in Trial 2. In Trial 1, baseline sweat rates during routine activities hovered around 2.8 nL min⁻¹ cm⁻² but elevated up to nearly 57 nL min⁻¹ cm⁻² during the presentation. Similarly, in Trial 2, baseline sweating rates were typically under 2.5 nL min⁻¹ cm⁻² but elevated to over 7.5 nL min⁻¹ cm⁻² while teaching. These trials demonstrate the capability of these patches to detect monitor the body's normal sweating response during routine activities over extended and full-day time periods, and from this identify when the body moves into physiologically deviating states such as those produced during stress. Many clinical tests of stress rely on self-reported and largely qualitative measures, but this work creates potential opportunities for continuous and quantitative stress testing through resting sweat rate.

Sweat Secretion Induced by Metabolic Changes.

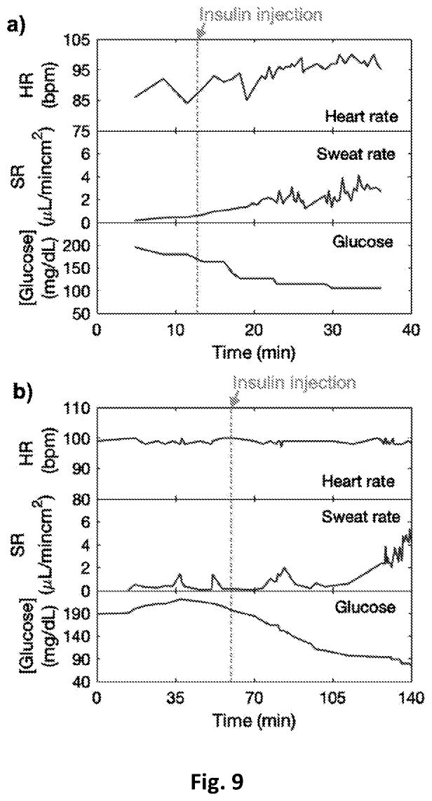

The patch was further utilized to investigate hypoglycemia-induced sweat secretion. In diabetic patients, injection of insulin gives rise to hyperhidrosis due to hypoglycemia^{43,44}. They can also be vulnerable to irregular heartbeat, which can be life-threatening⁴⁵. Understanding sweating and heart complications in diabetic patients, hence, can facilitate diabetes management. Toward this aim, we performed simultaneous monitoring of heart rate, sweat rate, and interstitial fluid (ISF) glucose levels to explore heart and sweat complications during large glucose variation. A diabetic subject wore the microfluidic patch on the finger along with a pulse oximeter. The measurement was done without interrupting the routine insulin injection procedures of the diabetic patient. During the measurement duration, the subject was asked to remain sitting without vigorous movements. ISF glucose data was recorded via Dexcom G6 continuous glucose monitor. FIG. 9, panels a, b shows measurements obtained from the two trials on the diabetic subject. In both trials, glucose was initially high when the measurement began, and the sweat rate remained relatively low between 0.5 and 1 μL min⁻¹ cm⁻². After insulin was injected, glucose started to decrease rapidly. In the meantime, an increase in sweat rate was observed. When glucose further decreased lower than 90 mg/dL in FIG. 9, panel b, there was a dramatic increase in sweat rate up to 5 μL min⁻¹ cm⁻². Heart rate remained relatively unchanged during low glucose level. Based on our results, significant decrease in glucose level is accompanied by a rise in sweat rate while no clear heart rate irregularity is observed. To develop this qualitative relation further, larger population studies must be conducted in future to quantitatively relate low glucose events and elevated sweating at rest.

Levodopa Sensing for Parkinson's Disease Management.

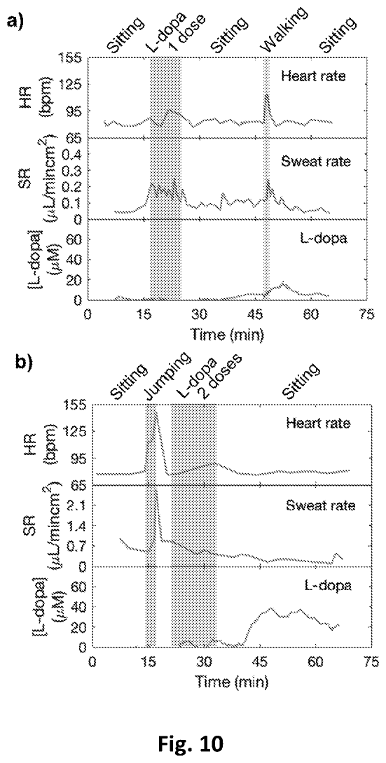

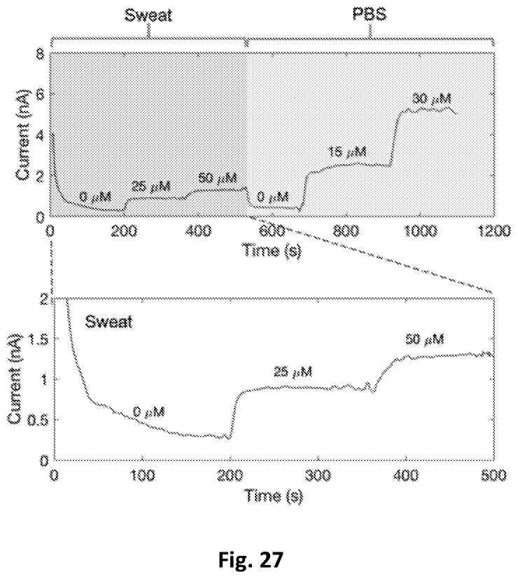

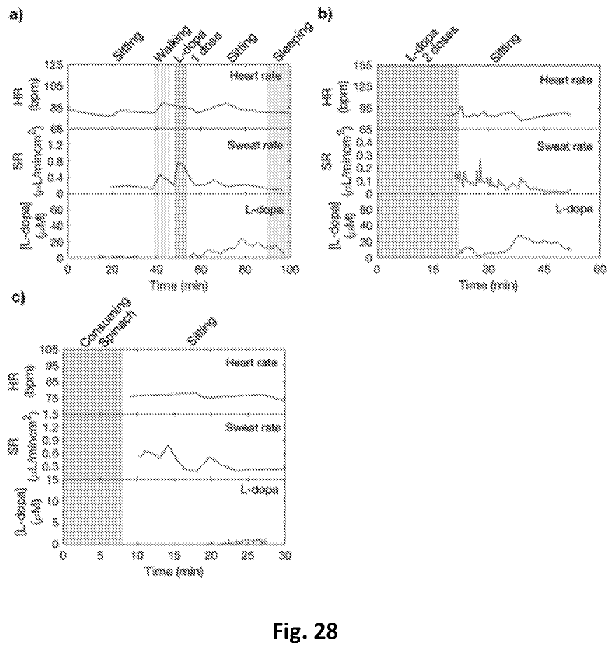

Levodopa is a first-line drug for treating Parkinson's disease. It has been reported that long-term intermittent oral dosage of levodopa causes fluctuation in plasma levodopa concentrations and leads to unpredictable responses such as motor fluctuations and dyskinesia; thus, continuous monitoring of levodopa is important to circumvent such unforeseen responses⁴⁶. Sweat has been reported to contain foreign drugs, including levodopa^{47,48}. Sweat is a promising noninvasive way to continuously monitor levodopa level inside the body. It may also facilitate finding an optimal dosage and interval that is personalized to each patient. In addition, Parkinson's patients usually suffer from abnormal sweating. Hyperhidrosis occurs when the blood levodopa concentration is low^{8,49} Therefore, studying sweat behavior and monitoring levodopa concentration can assist management of Parkinson's disease. Herein, we conducted on-body trials to study how sweat levodopa evolves within our body. A healthy subject was asked to consume 100 and 200 g intake of broad beans which contain levodopa⁵⁰ to observe sweat levodopa relation to broad beans intake. In this study, boiled broad beans which were reported to contain approximately 0.6 wt % levodopa were used⁵¹. This corresponds to levodopa intake similar to that of levodopa medication consumed by Parkinson's patients in a day. Levodopa sensors were calibrated in sweat as shown in FIG. 27 to ensure measurement accuracy and account for batch variation in absolute sensor signal. A sweat collection well of 3 mm diameter was used. In FIG. 10, panel a, it was observed that levodopa was detected in sweat approximately 20 min after initial intake and its concentration peaked at 35 min after intake. The peak concentration was measured to be approximately 13 μm when the subject had 1 dose of levodopa (1 dose of levodopa=100 g of broad beans). In FIG. 10, panel b, the subject again consumed 200 g of broad beans, and levodopa was measured approximately 20 min after initial intake. Its concentration peaked at 35 μm, 30 min after initial intake and slowly decreased. Additional trials presented in FIG. 28, panels a, b for 1 and 2 doses of levodopa intake showed similar results for the same subject. We observed that levodopa concentration in sweat generally increases with increasing doses. When other foods with minimal levodopa is consumed, no significant signal is observed (FIG. 28, panel c). This indicates that monitoring sweat levodopa may be a promising way to keep track of blood levodopa to assist medication management of Parkinson's disease patients. However, the exact relations between sweat levodopa concentration, plasma levels, and intake dose can depend on diet, hydration, other physiological conditions that impact absorption and metabolism rates, and on sweat rate and secretion mechanisms. Larger population studies must be performed to better understand the influence of these factors.

In summary, we present a wearable device for rapid uptake of nL min⁻¹ cm⁻² rates of thermoregulatory sweat at rest, enabling near-real-time sweat rate and composition analysis at rest. This represents a crucial advancement for detecting sweat rates associated with underlying physiological conditions, as demonstrated in subject studies exploring the relation between at-rest sweating and metabolic and stress conditions. Expanding on these preliminary trials, this patch can be deployed for patients or applications where deregulated sweating is a priori known to indicate underlying health conditions or can be used in exploratory subject studies to decode how sweating patterns relate to broader physiology. For example, hypoglycemia is known to qualitatively increase sweating rates as the body seeks to lower core temperature to conserve energy⁵². The presented patch can be used to more quantitatively study this phenomenon by simultaneously accumulating data on resting sweating rates and blood glucose levels, both for an individual over time and across a population of subjects. Personalized and universal correlations could then be built that enable resting sweat rate to serve as a noninvasive predictor of hypoglycemia. Similarly, excessive sweating is qualitatively known to indicate psychological duress, but more quantitative correlation studies can be performed between resting sweating rate and traditional, invasively obtained or discrete measures of mental state such as cortisol hormone levels⁵³. Based on these correlations, at-rest sweat rate could then be used to continuously and non-invasively estimate stress, with applications in assessing and improving the welfare of infants, soldiers, and stroke patients, and more generally of individuals going about everyday activities. More generally, the presented patch can be used to study correlations between sweat rates and composition, helping to better understand analyte secretion mechanisms and guide how measured concentrations should be interpreted. By allowing these studies to be performed in a way that is compatible with daily routines, this work creates fresh opportunities for decoding how noninvasive parameters relate to deeper body health and for establishing the physiological utility of sweat sensing as a whole.

### Methods.

Materials.

3-Aminopropyltriethoxysilane (APTES), polyvinyl butyral resin BUTVAR B-98 (PVB), aniline, sodium chloride, tyrosinase, glutaraldehyde, bovine serum albumin, thionine acetate salt, NAFION® 117, tetrabutylammonium bromide (TBAB), sodium chloride (NaCl) were purchased from Sigma-Aldrich. Aniline was distilled prior to usage. Silver ink CI-4040 was purchased from EMS Adhesives. Polydimethylsiloxane (Sylgard 184) was purchased from Ellsworth Adhesives. Moisture resistant polyester film 0.0005″ was purchased from McMasterCarr (Los Angeles, Calif.).

Sensor Fabrication.

Conductive Au electrodes were fabricated by standard photolithography and evaporation methods as detailed in our prior work²⁷. Electrochemical depositions required for sensor functionalization were performed on PCI4G300 (Gamry Instruments, USA). pH sensor was prepared by growing Au microstructures at 0 V for 30 s to roughen the surface as demonstrated in previous works⁵⁴, and then electrochemically depositing aniline solution (1 M HCl, 0.1 M aniline) by performing cyclic voltammetry from −0.2 to 1 V vs. Ag/AgCl at 100 mV/s for 25 cycles. Cl⁻ sensor was prepared by dropcasting silver ink and cured at 90° C. for 30 min. The electrode was subsequently treated with 0.1 M FeCl₃ for 1 min. The reference electrode for pH and Cl⁻ sensors was prepared by dropcasting a thin layer of silver ink onto the Au electrode. After drying, a solution containing 79.1 mg PVB and 50 mg NaCl in 1 mL methanol was dropcasted (10 pL/mm²). Levodopa sensor was prepared by initially growing Au nanodendrites using pulsed voltage from −1 to 1 V at a signal frequency of 50 Hz, 50% duty cycle, and 1500 cycles, creating high surface area structures as imaged in our previous work⁵⁵. Thionine acetate salt solution (0.25 mM) was deposited by applying 1 Hz signal frequency, pulsed voltage from −0.6 to 0 V, 90% duty cycle, and 660 cycles. Next, 0.2 pL of Tyrosinase solution containing 99 μL of 1% bovine serum albumin, 1 μL of 2.5% glutaraldehyde, and 0.25 μL of 1 mg/mL tyrosinase was dropcasted and dried. The membrane was additionally coated with 0.2 μL of NAFION-TBAB solution which was prepared as reported in literature⁵⁶. The levodopa sensors could be used after drying for an 30 hour at room temperature. For longterm storage, levodopa sensors were kept at 4° C. The shared reference/counter electrode for levodopa sensor was prepared by dropcasting silver ink and letting it dry before usage.

Microfluidic Device Fabrication.

Microfluidic was fabricated using standard photolithography process. SU8 photoresist was used to pattern microfluidics on a Si wafer. PDMS (base to curing agent ratio of 10:1) was poured onto the SU8 mold and cured at 60° C. for 4-5 h. The cured PDMS was peeled off and put under O₂ plasma, along with the PET patterned with sensing electrodes at a power of 90 W, 0.2 mtorr for 1 min. 1% APTES solution was dropcasted on entire surface of the PET for 2 min. The PET was cleaned with DI water and quickly dry with N₂. The PET was then bonded with PDMS and left it for at least an hour before usage. PDMS is soaked in DI water for 5 h prior to utilization to saturate PDMS⁵⁷ such that permeation-driven now is minimized⁵⁸. Oversaturation can also be achieved through longer presoak time at high temperature. By presoaking, sweat-containing microfluidic channel evaporated/diffused through the PDMS at 0.01 nL min⁻¹ cm⁻² when the device was tested for 8 h at 21-23° C. and relative humidity of 3942%.

Hydrophilic Filler Fabrication.

The patterned SU8 filler was prepared to a thickness of 200 μm on a flexible PET using standard procedures. The filler was carefully peeled off from the PET and put under O₂ plasma. A solution containing 0.5% PVA in DI water was then drop-casted onto the filler (0.5 μL/mm²), ensuring a complete coverage on the entire filler (including side and back walls), and was quickly heated on a hotplate at 80° C. The PVA film was approximately 10 μm in thickness. Once PVA dried, an AG-GLY film was placed on top of the filler. AG− GLY film was prepared by stirring and dissolving 2% agarose and 50% glycerol in DI water at 120° C. for 5 min. Once everything dissolved, ˜3 mL of the solution was quickly poured into a 100 mm hydrophilic glass dish and waited until the solution dried to become a gel-like film. The AG-GLY solution is viscous and dries easily; hence, rapid pour on a hydrophilic dish is necessary for a thin and uniform thickness. Here the AG-GLY film was not directly drop-casted on the filler because of the difficulty to achieve a thin uniform coating on the entire filler if we directly drop-casted the solution. The AG-GLY film was saturated with deionized water before placing on the filler. The film is approximately 90-130 μm thick. The laminated filler was finally placed inside the collection well of the microfluidic patch.

Device Characterization.

Sensor characterizations were performed on CHI1430 (CH Instruments, USA). The pH sensor was tested using McIlvaine's buffer of pH 4-8, and Cl⁻ sensor was tested using NaCl solution of concentration ranging from 25 to 200 mM. The potential difference with respect to a reference electrode was measured for both sensors. Levodopa sensor was measured by applying 0.35 V with respect to a shared reference/counter silver electrode. Flow rate experiments were carried out using Harvard Apparatus PHD 2000 Syringe Pump.

On-body sweat analysis. On-body human trials were carried out at the University of California, Berkeley in compliance with the human research protocol (CPHS 2014-08-6636 and CPHS 2015-05-7578) approved by the Berkeley Institutional Review Board (IRB). Both male and female subjects (between aged 21 and 45) were recruited from the Berkeley campus through campus flyers and verbal recruitments. Informed consents were obtained from all study subjects before enrollment in the study. The trials indicated in FIGS. 5 and 7, panel b were conducted at 20-23° C. and 39-50% relative humidity. Trials in FIG. 8 were conducted at 40-50% relative humidity with temperatures indicated in the figure. The trial in FIG. 10, panel b was conducted at 22° C. and 43% relative humidity. All other trials were conducted at 21° C. and 40% relative humidity. Targeted locations for sweat analysis were wiped with alcohol swab and gauze before application of the microfluidic device. Subjects were allowed to wear comfortable clothing. For heart rate measurements, a pulse oximeter (Zacurate Model 500DL) was used. The double-sided adhesive that was laminated between the skin and the patch was from Adhesive Research (93551). To ensure device could stay firmly on skin for the measurement durations, an irritation from these adhesives or prolonged patch wear, and no adhesive delamination, were found during the extended on-body trials, consistent with the adhesives' suitability of over 14 days of wear as stated by the manufacturer. For the on-body wrist sweat rate analysis, sweat rate sensors containing 24 radial electrodes were used. All the data presented were collected from separate measurements. Sweat composition data were collected using an electrochemical workstation CHI1430 (CH Instruments, USA). Electrical sweat rate data were collected using E4980AL precision LCR meter (Keysight Technologies). All the figures were plotted via Matlab.

Statistical Analysis.

Standard deviations shown in Fig. bookmark 12 5, panel c and reported in Table 2 are calculated by considering multiple measurements of instantaneous sweat rate at each tested body location.

### REFERENCES

1. Sonner, Z. et al. The microfluidics of the eccrine sweat gland, including biomarker partitioning, transport, and biosensing implications. Biomicrofluidics https://doi.org/10.1063/1.4921039 (2015).
2. Foster, K G, Hey, E N & O'Connell, B Sweat function in babies with defects of central nervous system. Arch. Dis. Child 46, 444-451 (1971).
3. Harpin, V A & Rutter, N Development of emotional sweating in the newborn infant. Arch. Dis. Child 57, 691-695 (1982).
4. Satoh, T., Ogawa, T. & Takagi, K. Sweating during daytime sleep. Jpn. J. Physiol. 15, 523-531 (1965).
5. Korpelainen, J. T., Sotaniemi, K. A. & Myllyla, V. V. Asymmetric sweating in stroke: a prospective quantitative study of patients with hemispheral brain infarction. Neurology 43, 1211-1214 (1993).
6. Wood, D. et al. Combat-related post-traumatic stress disorder: a case report using virtual reality graded exposure therapy with physiological monitoring with a female seabee. Mil. Med. 174, 1215-1222 (2009).
7. Cheshire, W. P. & Freeman, R. Disorders of sweating. Semin. Neurol. 23, 399-406 (2003).
8. Micieli, G., Tosi, P., Marcheselli, S. & Cavallini, A. Autonomic dysfunction in Parkinson's disease. Neurol. Sci. 24(Suppl 1), S32-S34 (2003).
9. Harker, M. Psychological sweating: a systematic review focused on aetiology and cutaneous response. Skin Pharm. Physiol. 26, 92-100 (2013).
10. Yang, Y. et al. A laser-engraved wearable sensor for sensitive detection of uric acid and tyrosine in sweat. Nat. Biotechnol. 38, 217-224 (2020).
11. Parlak, O., Keene, S. T., Marais, A., Curto, V. F. & Salleo, A. Molecularly selective nanoporous membrane-based wearable organic electrochemical device for noninvasive cortisol sensing. Sci. Adv. 4, eaar2904 (2018).
12. Lee, H. et al. Wearable/disposable sweat-based glucose monitoring device with multistage transdermal drug delivery module. Sci. Adv. 3, e1601314 (2017).
13. Yokus, M. A., Songkakul, T., Pozdin, V. A., Bozkurt, A. & Daniele, M. A. Wearable multiplexed biosensor system toward continuous monitoring of metabolites. Biosens. Bioelectron. 153, 112038 (2020).
14. Emaminejad, S. et al. Autonomous sweat extraction and analysis applied to cystic fibrosis and glucose monitoring using a fully integrated wearable platform. Proc. Natl Acad. Sci. USA 114, 4625-4630 (2017).
15. Jia, W. et al. Electrochemical tattoo biosensors for real-time noninvasive lactate monitoring in human perspiration. http://pubs.acs.org/doi/abs/10.1021/ac401573r (2013).
16. Kim, J. et al. Noninvasive alcohol monitoring using a wearable tattoo-based iontophoretic-biosensing system. http://pubs.acs.org/doi/abs/10.1021/acssensors.6b00356 (2016).
17. Nyein, H. Y. Y. et al. Regional and correlative sweat analysis using high-throughput microfluidic sensing patches toward decoding sweat. Sci. Adv. 5, eaaw9906 (2019).
18. Alizadeh, A. et al. A wearable patch for continuous monitoring of sweat electrolytes during exertion. Lab Chip 18, 2632-2641 (2018).
19. Bandodkar, A. J., Jeang, W. J., Ghaffari, R. & Rogers, J. A. Wearable sensors for biochemical sweat analysis. Annu Rev. Anal. Chem. 12, 1-22 (2019).
20. Li, S., Ma, Z., Cao, Z., Pan, L. & Shi, Y. Advanced wearable microfluidic sensors for healthcare monitoring. Small 16, 1903822 (2020).
21. Koh, A. et al. A soft, wearable microfluidic device for the capture, storage, and colorimetric sensing of sweat. Sci. Transl. Med. 8, 366ra165-366ra165 (2016).
22. Twine, N. B. et al. Open nanofluidic films with rapid transport and no analyte exchange for ultra-low sample volumes. Lab Chip 18, 2816-2825 (2018).
23. Bariya, M. et al. Roll-to-roll gravure printed electrochemical sensors for wearable and medical devices. ACS Nano 12, 6978-6987 (2018).
24. Lin, S. et al. Natural perspiration sampling and in situ electrochemical analysis with hydrogel micropatches for user-identifiable and wireless chemo/biosensing. ACS Sens. 5, 93-102 (2020).
25. Hauke, A. et al. Complete validation of a continuous and blood-correlated sweat biosensing device with integrated sweat stimulation. Lab Chip 18, 3750-3759 (2018).
26. Berger, M. J. & Kimpinski, K. Test-retest reliability of quantitative sudomotor axon reflex testing. J. Clin. Neurophysiol. 30, 308-312 (2013).
27. Nyein, H. Y. Y. et al. A wearable electrochemical platform for noninvasive simultaneous monitoring of Ca2+ and pH. http://pubs.acs.org/doi/abs/10.1021/acsnano.6b04005 (2016).
28. Tai, L.-C. et al. Wearable sweat band for noninvasive levodopa monitoring. Nano Lett. 19, 6346-6351 (2019).
29. Taylor, N. A. & Machado-Moreira, C. A. Regional variations in transepidermal water loss, eccrine sweat gland density, sweat secretion rates and electrolyte composition in resting and exercising humans. Extrem. Physiol. Med. 2, 4 (2013).
30. Bariya, M. et al. Glove-based sensors for multimodal monitoring of natural sweat. Sci. Adv. 6, eabb8308 (2020).
31. Hussain, J. N., Mantri, N. & Cohen, M. M. Working up a good sweat—the challenges of standardising sweat collection for metabolomics analysis. Clin. Biochem. Rev. 38, 13-34 (2017).
32. Koo, H.-J. & Velev, O. D. Design and characterization of hydrogel-based microfluidic devices with biomimetic solute transport networks. Biomicrofluidics 11, 024104 (2017).
33. Shay, T., Dickey, M. D. & Velev, O. D. Hydrogel-enabled osmotic pumping for microfluidics: towards wearable human-device interfaces. Lab Chip 17, 710-716 (2017).
34. Zhao, F. J. et al. Ultra-simple wearable local sweat volume monitoring patch based on swellable hydrogels. Lab Chip 20, 168-174 (2019).
35. Barrangou, L. M., Daubert, C. R. & Foegeding, E. A. Textural properties of agarose gels. I. Rheological and fracture properties. Food Hydrocoll. 20, 184-195.
36. Lee, H. et al. A graphene-based electrochemical device with thermoresponsive microneedles for diabetes monitoring and therapy. Nat. Nanotechnol. 11, 566-572 (2016).
37. Ogawa, T. Thermal influence on palmar sweating and mental influence on generalized sweating in man. Jpn. J. Physiol. 25, 525-536 (1975).
38. Kamei, T. et al. Physical stimuli and emotional stress-induced sweat secretions in the human palm and forehead. Anal. Chim. Acta 365, 319-326 (1998).
39. Shamsuddin, A. K. M. & Togawa, T. Continuous measurement of sweat electrolyte quantity to evaluate mental stress. in Proc. 18th Annual International Conference of the IEEE Engineering in Medicine and Biology Society. vol. 1 38-39 (1996).
40. Bahar, R. et al. The prevalence of anxiety and depression in patients with or without hyperhidrosis (HH). J. Am. Acad. Dermatol 75, 1126-1133 (2016).
41. Patterson, M. J., Galloway, S. D. R. & Nimmo, M. A. Variations in regional sweat composition in normal human males. Exp. Physiol. /core/journals/experimentalphysiology/article/div-classtitlevariations-in-regional-sweat-composition-in-normal-humanmalesdiv/31DAB66D8C207D90600B4CA48DDB1B89 (2000).
42. Allen, A. P. et al. The trier social stress test: principles and practice. Neurobiol. Stress 6, 113-126 (2016).
43. Passias, T. C., Meneilly, G. S. & Mekjavic, I. B. Effect of hypoglycemia on thermoregulatory responses. J. Appl. Physiol. 80, 1021-1032 (1996).
44. Buono, M. & Verity, L. Cholinergic-induced sweat rate during hypo- and hyperglycemia. Clin. Kinesiol. 58, 11-12 (2004).
45. Chow, E. et al. Risk of cardiac arrhythmias during hypoglycemia in patients with type 2 diabetes and cardiovascular risk. Diabetes 63, 1738-1747 (2014).
46. Olanow, C. W. et al. Continuous intrajejunal infusion of levodopa-carbidopa intestinal gel for patients with advanced Parkinson's disease: a randomised, controlled, double-blind, double-dummy study. Lancet Neurol. 13, 141-149 (2014).
47. Kintz, P., Henrich, A., Cirimele, V. & Ludes, B. Nicotine monitoring in sweat with a sweat patch. J. Chromatogr. B Biomed. Sci. Appl 705, 357-361 (1998).
48. Tsunoda, M., Hirayama, M., Tsuda, T. & Ohno, K. Noninvasive monitoring of plasma L-dopa concentrations using sweat samples in Parkinson's disease. Clin. Chim. Acta 442, 52-55 (2015).
49. Mano, Y., Nakamuro, T., Takayanagi, T. & Mayer, R. F. Sweat function in Parkinson's disease. J. Neurol. 241, 573-576 (1994).
50. Mehran, S. M., M. & B., G. Simultaneous determination of levodopa and carbidopa from fava bean, green peas and green beans by high performance liquid gas chromatography. J. Clin. Diagn. Res. 7, 1004-1007 (2013).
51. Etemadi, F., Hashemi, M., Randhir, R., ZandVakili, O. & Ebadi, A. Accumulation of 1-DOPA in various organs of faba bean and influence of drought, nitrogen stress, and processing methods on 1-DOPA yield. Crop J. 6, 426-434 (2018).
52. Kenny, G. P., Sigal, R. J. & McGinn, R. Body temperature regulation in diabetes. Temperature 3, 119-145 (2016).
53. Burke, H. M., Davis, M. C., Otte, C. & Mohr, D. C. Depression and cortisol responses to psychological stress: a meta-analysis. Psychoneuroendocrinology 30, 846-856 (2005).
54. Tian, Y., Liu, H., Zhao, G. & Tatsuma, T. Shape-controlled electrodeposition of gold nanostructures. J. Phys. Chem. B 110, 23478-23481 (2006).
55. Lin, Y. et al. Porous enzymatic membrane for nanotextured glucose sweat sensors with high stability toward reliable noninvasive health monitoring. Adv. Funct. Mater. 29, 1902521 (2019).
56. Meredith, S., Xu, S., Meredith, M. T. & Minteer, S. D. Hydrophobic salt-modified Nafion for enzyme immobilization and stabilization. J. Vis. Exp. https://doi.org/10.3791/3949 (2012).
57. Ojuroye, O., Torah, R. & Beeby, S. Modified PDMS packaging of sensory e-textile circuit microsystems for improved robustness with washing. Microsyst. Technol. https://doi.org/10.1007/s00542-019-04455-7 (2019).
58. Randall, G. C. & Doyle, P. S. Permeation-driven flow in poly (dimethylsiloxane) microfluidic devices. Proc. Natl Acad. Sci. USA 102, 10813-10818 (2005).

### Supplemental Materials.

Rationale for Microfluidic Channel Dimensions

The sweat gland is treated as a volumetric fluid source generating sweat at rate Q and exiting the sweat gland with secretory pressure P_{g}. This sweat is forced into the device with hydraulic resistance Rₜₒₜ=R_{well}+R_{channel}, producing a pressure drop of ΔP=RₜₒₜQ. To sustain this flow in the device, P_{g} must be larger than ΔP (ignoring atmospheric and Laplace pressures). P_{g} higher than this required pressure does not change the flow rate Q in the device but instead means that sweat will exit the microfluidic channel with some nonzero pressure.

Hydraulic resistance of the channel is given by

R_{channel}=12 μL/[0.37*w⁴]

where L=15 cm, w=70 um, and μ=viscosity=9.5*10⁻⁴ Pa-s.² By Darcy's law,

R_{well}=μL/kA=2.17*10¹³ Pa-s/m³

where k=Darcy permeability for 2% agarose hydrogels of 100 μm thickness≈620 nm²,³ L=100 μm, and A=cross sectional area of 3-mm diameter well.

Secretory pressures of the sweat gland for exercise and sauna-induced sweat are around 2.5 kPa, while those of chemically induced sweat can reach upwards of 70 kPa.^{1,4} Using the lower pressure as a conservative estimate, we can scale it down to lower resting sweat volumes (drawing on proportionalities indicated by Hoff's law P=σRTΔC since we expect the osmolality gradient to be related to secretion rate) assuming 2.5 kPa pressure corresponds conservatively to high rates of 20 nL min⁻¹ gland⁻¹. Then at extreme resting sweat rates of 1 nL min⁻¹ cm⁻² in a 3-mm diameter well, corresponding to 70.7 nL min⁻¹ entering the device, we can compare the estimated sweat gland secretory pressure of 1.2 kPa to the hydraulic pressure drops associated with different channel geometries to arrive at optimal dimensions (see, e.g., FIG. 30). We assume a square channel cross section and a channel length that overall allows us to hold around 750 nL in the channel.

Choosing a channel width and height of 70 μm and a length around 15 cm allows a large enough volume capacity as well as a cross sectional area that is small enough to ensure fast sweat speed in the channel (necessary for high-resolution sweat rate measurement) but large enough to avoid excessive hydraulic pressure losses. In this case Rₜₒₜ=R_{well}+R_{channel}=1.92*10¹⁴+2.17*10¹³ Pa-s/m³=2.1*10¹⁴ Pa-s/m³. ΔP is calculated for a broad range of resting sweat secretion and flow rates (high, medium, and low) and compared to the secretory pressure expected at those flow rates (according to P=2.5 kPa*Q/(20 nL min⁻¹ gland⁻¹)) (see, e.g. Table 3, in a 3-mm diameter region with 7 glands based on typical sweat gland densities of 100 glands cm⁻²) to confirm that the gland is a sufficient pump to inject sweat into a device of these dimensions.

TABLE 3
Parameters calculated for different secretion and flow rates.

| | | Q | | P | | ΔP | |
|---|---|---|---|---|---|---|---|
| | | (flow rate | | (secretory | | (hydraulic pressure | |
| Sweat secretion rate | | in device) | | pressure) | | loss in device) | |
| 1000 | nL/min-cm² | 70.7 | nL/min | 1.2 | kN/m² | 247 | N/m² |
| 50 | nL/min-cm² | 3.53 | nL/min | 63 | N/m² | 12.4 | N/m² |
| 3 | nL/min-cm² | 0.21 | nL/min | 3.8 | N/m² | 0.74 | N/m² |

Impact of Taylor Dispersion on Sensor Lag Times and Accuracy

Analyte diffusivity, sweat collection volume, and sweat secretion rate will impact the time lag between when sweat of a certain composition is secreted and when it is registered by the sensor. To estimate this, we consider sweat mixing and Taylor dispersion in the collection well and channel respectively using extremes of the above parameters. The following considerations are applied in our simulations:

1) The effective volume of the hydrogel-containing collection well is 72 nL for a 3 mm-diameter region. Because of the large-area proportions of the collection well, there is bulk mixing between older and fresher sweat that is treated as a continual averaging in the well.

2) We consider three sweat secretion rates (high—1000 nL min⁻¹ cm⁻², medium—50 nL min⁻¹ cm⁻², and low—3 nL min⁻¹ cm⁻²) that encompass a broad range of resting sweating rates. We consider sweat collection only in the 3 mm-diameter well as this broad range encompasses rates expected with the larger 8 mm opening. We consider the channel with cross section of 70 μm×70 μm.

3) Diffusivities of H⁺, Cl⁻, and levodopa fall between 1 and 10 (×10⁻⁹) m²/s in water and in the agarose hydrogel, so these extreme values are used in the simulations.^{5,6}

4) The concentration of sweat at the sensor position depends on older sweat deeper in the channel and on sweat upstream in the channel and well. Sensor accuracy thus depends on the specific sweat composition profile, but to give a general sense of the time lags involved we consider a step concentration profile in which sweat entering the channel has concentration 0.5 for t<0 and concentration of 1 at t≥0. We solve the diffusion-advection equation in 1D (along the channel length) while incorporating Taylor dispersion to consider the temporal accuracy with which our device can reconstruct this concentration profile.

Microchannel:

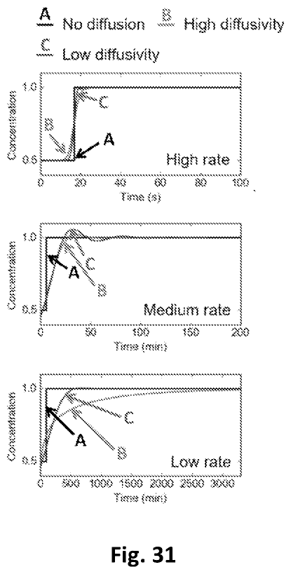

In the channel, the plots in FIG. 31 compare smearing out of the concentration transition step at the sensor location (0.4 cm into the channel) at the three different secretion rates and extreme diffusivities. Table 31 captures the time lag between when the step occurs at the entrance to the channel and is registered by the sensor as 90% of the complete step in concentration. Note that the time for the sensor to register this step without diffusion or dispersion is related to the sweat secretion rate and speed in the device, so the time to 90% reconstruction must be compared to this value.

TABLE 4
Time lag between when step occurs at the entrance
to the channel and is registered by the sensor
as 90% of the complete step in concentration.

| | | Time for sensor to | Time for sensor to | |
|---|---|---|---|---|
| Sweat secretion | | register step | register 90% of | |
| rate | Diffusivity | without diffusion | step with diffusion | |
| (nL/min-cm²) | (m²/sec) | or dispersion | and disperson | |
| 1000 | 1 × 10⁻⁹ | 16.6 min | 1.82 | s |
| | 10 × 10⁻⁹ | | 19.8 | s |
| 50 | 1 × 10⁻⁹ | 5.6 min | 20.24 | min |
| | 10 × 10⁻⁹ | | 22.4 | min |
| 3 | 1 × 10⁻⁹ | 92.4 min | 403.7 | min |
| | 10 × 10⁻⁹ | | 390.524 | min |

Collection Well:

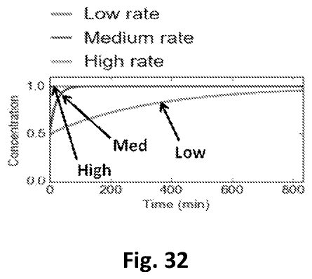

In the collection well, the plot below averages sweat at concentration 0.5 before t=0 with subsequent secretion of sweat at concentration 1 for t>0 for the three sweat rates. Table 5 below captures the time lag between when sweat at concentration 1 starts secreting and when the well captures 90% of the full change in concentration (see, FIG. 32. Note that this time lag is related to the rate of sweat secretion and the time for adequate replacement of earlier sweat in the well.

TABLE 5
The the time lag between when sweat at concentration
1 starts secreting and when the well captures
90% of the full change in concentration.

| Sweat secretion rate | Time for 90% chage in concentration with | |
|---|---|---|
| (nL/min-cm²) | diffusion and dispersion | |
| 1000 | 2.3 | min |
| 50 | 46.8 | min |
| 3 | 781 | min |

Overall, mixing and Taylor dispersion through the sections of the device indicate that at relatively high resting sweating rates on the fingertips, the sensor has a lag of around 3 minutes between when sweat at a certain composition is secreted and when it is detected at the sensor.

### SUPPLEMENTAL REFERENCES

1. Z. Sonner, et al. The microfluidics of the eccrine sweat gland, including biomarker partitioning, transport, and biosensing implications. Biomicrofluidics 9 (3), 031301 (2015).
2. Ojuroye O, Torah R, Beeby S. Modified PDMS packaging of sensory e-textile circuit microsystems for improved robustness with washing. Microsyst Technol [Internet]. 2019 May 18 [cited 2020 Oct. 29]; Available from: https://doi.org/10.1007/s00542-019-04455-7.
3. E. M. Johnson, W. M. Deen. Hydraulic permeability of agarose gels. AlChE Journal 42 (5), 1220-1224 (1996).
4. J. Choi, et al. Soft, skin-mounted microfluidic systems for measuring secretory fluidic pressures generated at the surface of the skin by eccrine sweat glands. Lab on a Chip 17 (15), 2572-2580 (2017).
5. M. Safaei, et al. Electrochemical Sensing of Levodopa in Presence of Tryptophan Using Modified Graphite Screen Printed Electrode with Magnetic Core-Shell Fe 3 O 4@ SiO 2/GR Nanocomposite. Surface Engineering and Applied Electrochemistry 56, 184-191 (2020).
6. G. Schuszter, et al. Determination of the diffusion coefficient of hydrogen ion in hydrogels. Phys. Chem. 19 (19), 12136-12143 (2017).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A wearable biometric monitoring system comprising:
a hydrophilic material 106;
a sensing electrode 104; and
a microfluidic channel 110 connecting said hydrophilic material and said sensing electrode.

2. The wearable biometric monitoring system of claim 1, wherein device comprises a collection well 108 in fluid communication with said microfluidic channel and said hydrophilic material 106 is disposed in said collection well.

3. The wearable biometric monitoring system according to any one of claims 1-2, wherein said collection well provides a collection area ranging in diameter from about 1 mm to about 20 mm, or from about 2 mm up to about 10 mm, or from about 3 mm up to about 7 mm.

4. The wearable biometric monitoring system according of claim 3, wherein said collection well provides a collection area of about 8 mm.

5. The wearable biometric monitoring system according of claim 3, wherein said collection well provides a collection area of about 5 mm.

6. The wearable biometric monitoring system according of claim 3, wherein said collection well provides a collection area of about 3 mm.

7. The wearable biometric monitoring system according to any one of claims 1-6, wherein said hydrophilic material is laminated and includes hydrogel 204.

8. The wearable biometric monitoring system according to any one of claims 1-7, wherein said hydrogel comprises an agarose-glycerol (AG-GLY) hydrogel.

9. The wearable biometric monitoring system according to any one of claims 1-8, wherein said hydrophilic material comprises a hydrophilic polymer disposed on a patterned substrate.

10. The wearable biometric monitoring system of claim 9, wherein said hydrophilic polymer comprises polyvinyl alcohol (PVA).

11. The wearable biometric monitoring system according to any one of claims 1-10, wherein said patterned substrate comprises a patterned epoxy substrate.

12. The wearable biometric monitoring system of claim 11, wherein said substrate comprises a patterned SU8 substrate.

13. The wearable biometric monitoring system according to any one of claims 1-12, wherein said hydrophilic material comprises laminated substrate comprising a hydrophilic polymer disposed on a patterned substrate that is coated with a hydrophilic polymer.

14. The wearable biometric monitoring system according to any one of claims 1-13, wherein said microfluidic channel has a length of less than 33 cm, or less than 30 cm, or less than 25 cm, or less than 20 cm, or about 15 cm or less.

15. The wearable biometric monitoring system according to any one of claims 1-14, wherein said microfluidic channel has a minimum volume of about 750 nL.

16. The wearable biometric monitoring system according to any one of claims 14-15, wherein said microfluidic channel has a length of about 15 cm or less.

17. The wearable biometric monitoring system according to any one of claims 1-16, wherein said microfluidic channel has dimensions that provide a flow rate drop of less than about 10% along the length of said microfluidic channel.

18. The wearable biometric monitoring system according to any one of claims 1-17, wherein said microfluid channel has a cross-section area at least about 2,209 μm² (e.g., 47 μm×47 μm), or at least about 3600 μm², or at least about 4900 μm² (e.g., 70 μm×70 μm), or at least about 700 μm², or at least about 14,000 μm² (e.g., 200 μm×70 μm).

19. The wearable biometric monitoring system of claim 18, wherein said microfluidic channel has a cross-section area of about 70 μm×70 μm.

20. The wearable biometric monitoring system of claim 18, wherein said microfluid channel has a cross-section area of about 200 μm×70 μm.

21. The wearable biometric monitoring system according to any one of claims 1-20, wherein said sensing electrode(s) 104 are configured to be in fluid communication with a fluid in said microfluidic channel.

22. The wearable biometric monitoring system of claim 21, wherein said sensing electrodes 104 are configured to be aligned with the microfluidic channel 110.

23. The wearable biometric monitoring system according to any one of claims 21-22, wherein said sensing electrodes 104 are configured as two interdigitated wheel-shaped electrodes aligned with the microfluidic channel 110.

24. The wearable biometric monitoring system according to any one of claims 21-23, where said sensing electrodes comprise sweat rate sensing electrode(s) 104a and analyte detecting electrodes 104b.

25. The wearable biometric monitoring system of claim 24, wherein said sweat rate sensing electrodes 104a comprise radial conductive electrodes 104a1.

26. The wearable biometric monitoring system according to any one of claims 24-25, wherein said analyte detecting electrodes 104b comprise one or more regions 104b1 functionalized for detection of pH and/or an analyte.

27. The wearable biometric monitoring system of claim 26, wherein said analyte detecting electrode(s) 104b are functionalized for detection and/or quantification of an analyte selected from the group consisting of a metabolite, a drug, ethanol, a metal ion, and a salt.

28. The wearable biometric monitoring system according to any one of claims 1-27, wherein sensing electrode 104 is configured to measure sweat rate.

29. The wearable biometric monitoring system according to any one of claims 1-28, wherein sensing electrode 104 is configured to measure pH, Cl⁻, and/or levodopa.

30. The wearable biometric monitoring system of claim 29, wherein said system measures pH.

31. The wearable biometric monitoring system of claim 29, wherein said system measures Cl⁻.

32. The wearable biometric monitoring system of claim 29, wherein said system measures levodopa.

33. The wearable biometric monitoring system according to any one of claims 1-32, wherein said system is configured for detection by detection and/or quantification of electrical current or electrical potential.

34. The wearable biometric monitoring system according to any one of claims 1-33, wherein said microfluidic channel 110 is disposed in a microfluidic chip 102.

35. The wearable biometric monitoring system according to any one of claims 1-20, wherein said device is disposed on a flexible substrate 112.

36. The wearable biometric monitoring system of claim 35, wherein said substrate a flexible polymer.

37. The wearable biometric monitoring system of claim 36, wherein said substrate comprises polyethylene terephthalate (PET).

38. The wearable biometric monitoring system according to any one of claims 1-37, wherein said wearable biometric monitoring system comprises a skin adhesive 114 compatible with application to the skin.

39. The wearable biometric monitoring system of claim 38, wherein said skin adhesive 114 is disposed so that when said device is attached to the skin of a subject, said collection well is juxtaposed against a surface of said skin.

40. A wearable patch for analysis of a user's sweat comprising:
skin adhesive;
a microfluidic chip with a hydrophilic material and a microfluidic channel;
a sensing electrode;
wherein said skin adhesive is capable of attaching said microfluidic chip to the skin of a user and said hydrophilic material is capable of drawing sweat from said user so that said sweat can be transported into said microfluidic channel and to said electrode for analysis.

41. The wearable patch of claim 6 wherein said sensing electrode measures sweat rate.

42. The wearable patch of claim 6 wherein said sensing electrode is an electrochemical sensor which senses pH, Cl and/or levodopa.

43. A method of analyzing a user's sweat comprising:
selecting a patch comprising a skin adhesive, a microfluidic chip with a hydrophilic material, a microfluidic channel and a sensing electrode;
using said adhesive to apply said patch to a user's skin; and
collecting sweat from said user by drawing sweat from said user's skin with said hydrophilic material and transporting said sweat to said sensing electrode through said microfluidic channel; and, using said sensing electrode to analyze said user's sweat.

44. A method of analyzing a subject's sweat, said method comprising:
providing a subject with a wearable biometric monitoring system according to any one of claims 1-39 attached to the surface of the skin of said subject; and
operating said monitoring system to analyze the sweat of said subject.

45. The method of claim 44, wherein said monitoring system is operated to detect the sweat rate of said subject.

46. The method according to any one of claims 44-45, wherein said monitoring system is operated to determine the pH of the sweat of said subject.

47. The method according to any one of claims 44-46, wherein said monitoring system is operated to detect an analyte in the sweat of said subject where said analyte is selected from the group consisting of a metabolite, a drug, ethanol, a metal ion, and a salt.

48. The method of claim 47, wherein said monitoring system is operated to detect and/or quantify pH, Cl⁻, and/or levodopa in the sweat of said subject.

49. The method of claim 48, wherein said monitoring system is operated to measure Cl⁻ in the sweat of said subject.

50. The method of claim 48, wherein said monitoring system is operated to measure levodopa in the sweat of said subject.

51. The method according to any one of claims 44-50, wherein said subject is a human.

52. The method according to any one of claims 44-50, wherein said subject is a non-human mammal.
